(19) United States
(12) Patent Application Publication
(10) Pub. No.: US 2026/0301468 A1
(43) Pub. Date: Oct. 1, 2026

(54) COMPUTER VISION FUNCTIONALITIES FOR INDIVIDUAL AND/OR GROUP SESSION

(71) Applicant: BALLOGY, INC., Austin, TX (US)

(72) Inventors: Todd Young, Austin, TX (US); Syed Saad Hussain, Lahore (PK); Jordan Green, Austin, TX (US)

(21) Appl. No.: 19/461,576

(22) Filed: Jan. 27, 2026

Related U.S. Application Data
(60) Provisional application No. 63/750,261, filed on Jan. 27, 2025.

(51) Int. Cl. G06V 40/20; G06F 3/04842

(52) U.S. Cl. CPC G06V 40/23; G06F 3/04842

(57) ABSTRACT

Briefly, example methods, apparatuses, and/or articles of manufacture are disclosed that may be implemented, in whole or in part, via one or more computing and/or communication networks and/or protocols using one or more electronic devices to facilitate and/or support one or more computer vision operations and/or techniques, such as for athletic and/or skills assessment, development, and/or performance, which may include electronically generating and/or tracking athletic training workouts for individual users and/or groups.

The present patent application claims priority from U.S. provisional patent application Ser. No. 63/750,261, entitled “COMPUTER VISION FUNCTIONALITIES FOR INDIVIDUAL AND/OR GROUP SESSION,” filed Jan. 27, 2025, the contents of which are incorporated herein by reference in their entirety and for all purposes.

### BACKGROUND

### Field

The present disclosure relates generally to computer vision infrastructures and, more particularly, to computer vision infrastructures and related functionalities (e.g., operations, techniques, processes, etc.), such as implemented, in whole or in part, via electronic devices in connection with athletic and/or skills assessment, development, and/or performance, which may include electronically generating and/or tracking athletic training workouts for individual users and/or groups.

### BRIEF DESCRIPTION OF THE DRAWINGS

Claimed subject matter is particularly pointed out and distinctly claimed in the concluding portion of the specification. However, both as to organization and/or method of operation, together with objects, features, and/or advantages thereof, it may best be understood by reference to the following detailed description if read with the accompanying drawings in which:

FIG. 1 is a schematic block diagram depicting an embodiment of an example system including one or more server computing devices and/or one or more IoT-type devices;

FIG. 2 is a schematic block diagram depicting an embodiment of an example Internet of Things (IoT)-type device;

FIG. 3 is an illustration depicting an overall view of example computer vision functionalities explored in more detail in FIGS. 4A-4O, in accordance with one or more embodiments;

FIGS. 4A-4O depict aspects of a sports social media application pertaining, at least in part, to computer vision operations, processes, etc., in accordance with one or more embodiments;

FIG. 5 depicts a flow diagram illustrating an example process for athletic and/or skills assessment, development, and/or performance via computer vision operations, processes, etc., in accordance with one or more embodiments;

FIG. 6 depicts a schematic diagram illustrating an implementation of an example computing environment, in accordance with one or more embodiments;

FIG. 7 is a schematic diagram of a neural network formed in “layers”, in accordance with one or more embodiments; and

FIG. 8 is a flow diagram of an aspect of a neural network training operation, in accordance with one or more embodiments.

Reference is made in the following detailed description to accompanying drawings, which form a part hereof, wherein like numerals may designate like parts throughout that are corresponding and/or analogous. It will be appreciated that the figures have not necessarily been drawn to scale, such as for simplicity and/or clarity of illustration. For example, dimensions of some aspects may be exaggerated relative to others. Further, it is to be understood that other embodiments may be utilized. Furthermore, structural and/or other changes may be made without departing from claimed subject matter. References throughout this specification to “claimed subject matter” refer to subject matter intended to be covered by one or more claims, or any portion thereof, and are not necessarily intended to refer to a complete claim set, to a particular combination of claim sets (e.g., method claims, apparatus claims, etc.), or to a particular claim. It should also be noted that directions and/or references, for example, such as up, down, top, bottom, and so on, may be used to facilitate discussion of drawings and are not intended to restrict application of claimed subject matter. Therefore, the following detailed description is not to be taken to limit claimed subject matter and/or equivalents. It should also be noted that “subject matter” and “claimed subject matter” can be used interchangeably herein.

### DETAILED DESCRIPTION

References throughout this specification to one implementation, an implementation, one embodiment, an embodiment, and/or the like means that a particular feature, structure, characteristic, and/or the like described in relation to a particular implementation and/or embodiment is included in at least one implementation and/or embodiment of claimed subject matter. Thus, appearances of such phrases, for example, in various places throughout this specification are not necessarily intended to refer to the same implementation and/or embodiment or to any one particular implementation and/or embodiment. Furthermore, it is to be understood that particular features, structures, characteristics, and/or the like described are capable of being combined in various ways in one or more implementations and/or embodiments and, therefore, are within intended claim scope. In general, of course, as has always been the case for the specification of a patent application, these and other issues have a potential to vary in a particular context of usage. In other words, throughout the patent application, particular context of description and/or usage provides helpful guidance regarding reasonable inferences to be drawn; however, likewise, “in this context” in general without further qualification refers to the context of the present patent application.

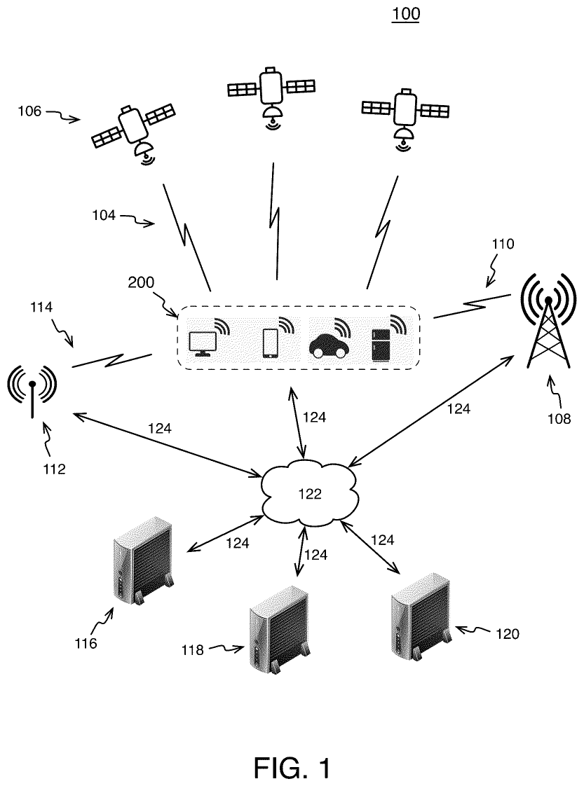

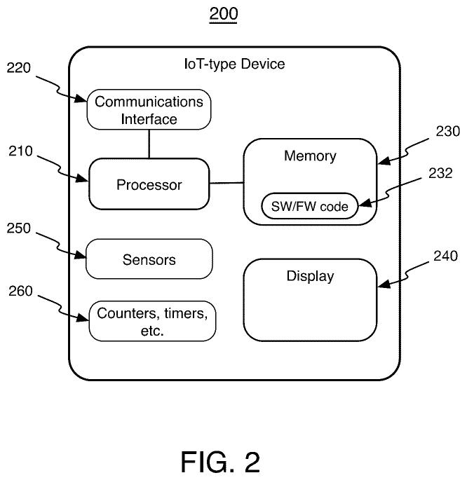

Even though a number of particular implementations are described herein, it should be noted, however, that subject matter is not limited in scope to the particular example implementations provided. Also, example implementations may utilize, at least in part, any of a wide range of computing and/or communication devices, systems, components, technologies, networks, etc. The discussion below, including aspects related to FIGS. 1 and 2, for example, describes various aspects of an example infrastructure that may facilitate and/or support, at least in part, one or more example implementations discussed herein.

The “World Wide Web” or simply the “Web,” such as provided by the Internet, for example, is growing rapidly, at least in part, from the large amount of content being added seemingly on a daily basis. A wide variety of content in the form of stored signals, such as, for example, text files, images, audio files, video files, web pages, measurements of physical phenomena, and/or the like may be continually acquired, identified, located, retrieved, collected, stored, communicated, etc. Increasingly, content is being acquired, collected, communicated, etc. by a number of electronic devices, such as, for example, embedded computing devices leveraging existing Internet and/or like infrastructure as part of a so-called “Internet of Things” (IoT), such as via a variety of protocols, domains, and/or applications. IoT may typically comprise a system of interconnected and/or internetworked physical computing devices capable of being identified, such as uniquely via an assigned Internet Protocol (IP) address, for example. Devices, such as IoT-type devices, for example, may include computing resources embedded into hardware so as to facilitate and/or support a device's ability to acquire, collect, process and/or transmit content over one or more communications networks. In this context, “IoT-type devices” and/or the like refer to one or more electronic and/or computing devices capable of leveraging existing Internet and/or like infrastructure as part of the IoT, such as via a variety of applicable protocols, domains, applications, etc. In particular implementations, IoT-type devices, for example, may comprise a wide variety of embedded devices, such as, for example, automobile sensors, biochip transponders, heart monitoring implants, thermostats, kitchen appliances, locks or like fastening devices, solar panel arrays, home gateways, controllers, etc. and even mobile devices, desktop computers, laptop computers, and/or the like. Although embodiments described herein may refer to IoT-type devices, subject matter is not limited in scope in these respects. For example, although IoT-type devices may be described, such as for ease of discussion, it should be noted that subject matter is intended to include use of any of a wide range of electronic device types, including a wide range of computing and/or communications device types.

“Electronic content,” “digital content,” “content,” and/or the like as the terms are used herein should be interpreted broadly and refers to signals, such signal packets, for example, and/or states, such as physical states on a memory device, for example, but otherwise are employed in a manner irrespective of format, such as any expression, representation, realization, and/or communication, for example. Content may comprise, for example, any information, knowledge, and/or experience, such as, again, in the form of signals and/or states, physical or otherwise. In this context, “electronic” or “on-line” content refers to content in a form that although not necessarily capable of being perceived by a human, (e.g., via human senses, etc.) may nonetheless be transformed into a form capable of being so perceived, such as visually, haptically, and/or audibly, for example. Non-limiting examples may include text, audio, images, video, security parameters, combinations, or the like. Thus, content may be stored and/or transmitted electronically, such as before or after being perceived by human senses. In general, it may be understood that electronic content may be intended to be referenced in a particular discussion, although in the particular context, the term “content” may be employed for ease of discussion. Specific examples of content may include, for example, computer code, data, metadata, message, text, audio file, video file, data file, web page, or the like. Claimed subject matter is not intended to be limited to these particular examples, of course.

FIG. 1 is a schematic diagram illustrating features associated with an implementation of an example operating environment 100 capable of facilitating and/or supporting one or more operations and/or techniques for automatic assignment-type curriculum Workout plans, Featured Workouts, team training, skills assessment, etc., discussed more fully below, for example, for distributed and/or remote computing and/or communications networks, illustrated generally herein at 200. As was indicated, one or more operations and/or techniques may, for example, be implemented. At least in part, in connection with one or more IoT-type devices, though subject matter is not so limited. Briefly, IoT is typically a system of interconnected and/or internetworked physical devices in which computing may be embedded into hardware so as to facilitate and/or support devices' abilities to acquire, collect and/or communicate content over one or more communications networks, for example, at times, without human participation and/or interaction. As mentioned, IoT-type devices may include a wide variety of stationary and/or mobile devices, such as, for example, automobile sensors, biochip transponders, heart monitoring implants, kitchen appliances, locks or like fastening devices, solar panel arrays, home gateways, smart gauges, smart telephones, cellular telephones, security cameras, wearable devices, thermostats, Global Positioning System (GPS) transceivers, personal digital assistants (PDAs), virtual assistants, laptop computers, personal entertainment systems, tablet or other personal computers (PCs), personal audio and/or video devices, personal navigation devices, mobile devices, and/or the like.

It should be appreciated that operating environment 100 is described herein as a non-limiting example that may be implemented, in whole or in part, in a context of various wired and/or wireless communications networks and/or any suitable portion and/or combination of such networks. For example, these or like networks may include one or more public networks (e.g., the Internet, the World Wide Web), private networks (e.g., intranets), wireless wide area networks (WWAN), wireless local area networks (WLAN, etc.), wireless personal area networks (WPAN), telephone networks, cable television networks, Internet access networks, fiber-optic communication networks, waveguide communication networks and/or the like. It should also be noted that claimed subject matter is not limited to a particular network and/or operating environment. Thus, for a particular implementation, one or more operations and/or techniques for automatic assignment-type curriculum Workout plans for distributed computing and/or communications networks may be performed, at least in part, in an indoor environment and/or an outdoor environment, or any combination thereof.

Thus, as illustrated, in a particular implementation, one or more computing and/or communications devices, such as IoT-type devices 200 may, for example, receive and/or acquire satellite positioning system (SPS) signals 104 from SPS satellites 106. In some instances, SPS satellites 106 may be from a single global navigation satellite system (GNSS), such as the GPS or Galileo satellite systems, for example. In other instances, SPS satellites 106 may be from multiple GNSS such as, but not limited to, GPS, Galileo, Glonass, or Beidou (Compass) satellite systems, for example. In certain implementations, SPS satellites 1006 may be from any one several regional navigation satellite systems (RNSS) such as, for example, WAAS, EGNOS, QZSS, just to name a few examples.

At times, one or more IoT-type devices 200 may, for example, transmit wireless signals to and/or receive wireless signals from a suitable wireless communication network. In one example, one or more IoT-type devices 200 may communicate with a cellular communication network, such as by transmitting wireless signals to and/or receiving wireless signals from one or more wireless transmitters capable of transmitting and/or receiving wireless signals, such as a base station transceiver 108 over a wireless communication link 110, for example. Similarly, one or more IoT-type devices 200 may transmit wireless signals to and/or receive wireless signals from a local transceiver 112 over a wireless communication link 114, for example. Base station transceiver 108, local transceiver 112, etc. may be of the same or similar type, for example, and/or may represent different types of devices, such as access points, radio beacons, cellular base stations, femtocells, an access transceiver device, or the like, depending on an implementation. Similarly, local transceiver 112 may comprise, for example, a wireless transmitter and/or receiver capable of transmitting and/or receiving wireless signals. For example, at times, wireless transceiver 112 may be capable of transmitting and/or receiving wireless signals from one or more other terrestrial transmitters and/or receivers.

In a particular implementation, local transceiver 112 may, for example, be capable of communicating with one or more IoT-type devices 200 at a shorter range over wireless communication link 114 than at a range established via base station transceiver 108 over wireless communication link 110. For example, local transceiver 112 may be positioned in an indoor or like environment and/or may provide access to a wireless local area network (WLAN, e.g., IEEE Std. 802.11 network, etc.) and/or wireless personal area network (WPAN, e.g., Bluetooth® network, etc.). In another example implementation, local transceiver 112 may comprise a femtocell and/or picocell capable of facilitating communication via link 114 according to an applicable cellular or like wireless communication protocol. Again, it should be understood that these are merely examples of networks that may communicate with one or more IoT-type devices 200 over a wireless link, and claimed subject matter is not limited in this respect. For example, in some instances, operating environment 100 may include a larger number of base station transceivers 108, local transceivers 112, networks, terrestrial transmitters and/or receivers, etc.

In an implementation, one or more IoT-type devices 200, base station transceiver 108, local transceiver 112, etc. may, for example, communicate with one or more servers, referenced herein at 116, 118, and 120, over a network 122, such as via one or more communication links 124. It should be noted that, depending on an implementation, one or more servers 116, 118, and/or 120 may be part of a centralized network, a decentralized network, or any combination thereof. Thus, even though terms like “server” or “servers” are used herein, such as for ease of discussion, it should be appreciated that these or like aspects may include and/or be part of one or more centralized and/or decentralized networks. Thus, as indicated, network 122 may comprise, for example, a centralized network, a decentralized network, or any combination thereof, and may include any number and/or combination of wired and/or wireless communication links. In a particular implementation, network 122 may comprise, for example, Internet Protocol (IP)-type infrastructure capable of facilitating or supporting communication between one or more IoT-type devices 200 and one or more servers 116, 118, 120, etc. via local transceiver 112, base station transceiver 108, directly, etc. In another implementation, network 122 may comprise, for example cellular communication network infrastructure, such as a base station controller and/or master switching center to facilitate and/or support mobile cellular communication with one or more IoT-type devices 200. Servers 116, 118 and/or 120 may comprise any suitable servers or combination thereof capable of facilitating or supporting one or more operations and/or techniques discussed herein. For example, servers 116, 118 and/or 120 may comprise one or more update servers, back-end servers, management servers, archive servers, location servers, positioning assistance servers, navigation servers, map servers, crowdsourcing servers, network-related servers, or the like.

Even though a certain number of computing platforms and/or devices are illustrated herein, any number of suitable computing platforms and/or devices may be implemented to facilitate and/or support one or more techniques and/or processes associated with operating environment 100. For example, at times, network 122 may be coupled to one or more wired and/or wireless communication networks (e.g., WLAN, etc.) so as to enhance a coverage area for communications with one or more IoT-type devices 200, one or more base station transceivers 108, local transceiver 112, servers 116, 118, 120, or the like. In some instances, network 122 may facilitate and/or support femtocell-based operative regions of coverage, for example. Again, these are merely example implementations, and claimed subject matter is not limited in this regard.

In this context, “IoT-type devices” refer to one or more electronic and/or computing devices capable of leveraging existing Internet or like infrastructure as part of the so-called “Internet of Things” or IoT, such as via a variety of applicable protocols, domains, applications, etc. As was indicated, the IoT is typically a system of interconnected and/or internetworked physical devices in which computing may be embedded into hardware so as to facilitate and/or support devices' ability to acquire, collect, and/or communicate content over one or more communications networks, for example, at times, without human participation and/or interaction. IoT-type devices 200, for example, may include a wide variety of stationary and/or mobile devices, such as, for example, automobile sensors, biochip transponders, heart monitoring implants, kitchen appliances, locks or like fastening devices, solar panel arrays, home gateways, smart gauges, smart telephones, cellular telephones, security cameras, wearable devices, thermostats, Global Positioning System (GPS) transceivers, personal digital assistants (PDAs), virtual assistants, laptop computers, personal entertainment systems, tablet personal computers (PCs), PCs, personal audio or video devices, personal navigation devices, mobile devices, stationary devices, and/or the like, to name a few non-limiting examples. Typically, in this context, a “mobile device” refers to an electronic and/or computing device that may from time to time have a position or location that changes, and/or a “stationary device” refers to an electronic and/or computing device that may have a position or location that generally does not change. In some instances, IoT-type devices, such as IoT-type devices 200, may be capable of being identified, such as uniquely, via an assigned Internet Protocol (IP) address (e.g., static, dynamic, etc.), as one particular example, and/or having an ability to communicate, such as receive and/or transmit electronic content, for example, over one or more wired and/or wireless communications networks.

It may again be noted that the example infrastructure discussed above, along with additional systems, apparatuses, processes, etc., discussed herein, may be directed, at least in part, to supporting automatic assignment-type curriculum Workout plans, for example. The example infrastructure discussed above, along with additional systems, apparatuses, processes, etc., discussed herein, may be further directed, at least in part, to helping coaches and/or parents, for example, to guide players, such as basketball players, in their development of skills, techniques, strategies, etc., associated with the game of basketball. As discussed more fully below, coaches, players, parents, other group owners and/or individuals may electronically communicate with other coaches, players, parents, other group owners and/or individuals and/or with one or more administrator computing devices via an application, or portions of an application, executed at least in part on their respective electronic devices. The example infrastructure described in connection with FIG. 1 and FIG. 2, for example, may provide a platform to support example functionalities described herein, including automatic assignment-type curriculum Workout plans, described in more detail below. It may also be appreciated that one or more software and/or firmware agents implemented to facilitate example functionalities described herein, including automatic assignment-type curriculum Workout plans, for example, may be partitioned between administrator computing devices, such as servers 120, for example, and any number of remote and/or mobile devices, such as IoT-type devices 200, for example, that may be associated with one or more coaches, players, parents, etc.

FIG. 2 is an illustration of an embodiment 200 of an example particular IoT-type device. Of course, subject matter is not limited in scope to the particular configurations and/or arrangements of components depicted and/or described for example devices mentioned herein. In an embodiment, an IoT-type device, such as 200, may comprise one or more processors, such as processor 210, and/or may comprise one or more communications interfaces, such as communications interface 220. In an embodiment, one or more communications interfaces, such as communications interface 220, may enable wireless and/or wired communications between an electronic device, such as an IoT-type device 200, and one or more other computing devices. In an embodiment, wireless and/or wired communications may occur substantially in accordance any of a wide range of communication protocols, such as those known and/or mentioned herein, for example, and/or developed in the future.

In a particular implementation, an IoT-type device, such as IoT-type device 200, may include a memory, such as memory 230. In a particular implementation, memory 230 may comprise a non-volatile memory, for example. Further, in a particular implementation, a memory, such as memory 230, may have stored therein executable instructions, such as for one or more operating systems, communications protocols, and/or applications, for example. A memory, such as 230, may further store particular instructions, such as software and/or firmware code 232, that may be updated via one or more example implementations and/or embodiments described herein. Further, in a particular implementation, an IoT-type device, such as IoT-type device 200, may comprise a display, such as display 240, and/or one or more sensors, such as one or more sensors 250. As utilized herein, “sensors” and/or the like refer to a device and/or component that may respond to physical stimulus, such as, for example, heat, light, sound pressure, magnetism, particular motions, etc., and/or that may generate one or more signals and/or states in response to physical stimulus. Example sensors may include, but are not limited to, one or more accelerometers, gyroscopes, thermometers, magnetometers, barometers, light sensors, proximity sensors, hear-rate monitors, perspiration sensors, hydration sensors, breath sensors, cameras, microphones, etc., and/or any combination thereof. It may be noted that IoT-type devices, such as device 200, may include one or more cameras to facilitate video recording, for example.

In particular implementations, IoT-type device 200 may include one or more timers and/or counters and/or like circuits, such as circuitry 260, for example. In an embodiment, one or more timers and/or counters and/or the like may track one or more aspects of device performance and/or operation. For example, timers, counters, and/or other like circuits may be utilized, at least in part, by IoT-type device 200 to determine measures of fitness, for example, and/or to otherwise generate feedback content related to testing results, in particular implementations.

Although FIG. 2 depicts a particular example implementation of an IoT-type device, such as IoT-type device 200, other embodiments may include other types of electronic and/or computing devices. Example types of electronic and/or computing devices may include, for example, any of a wide range of digital electronic devices, including, but not limited to, cellular telephones (e.g., smartphones), tablet devices, desktop and/or notebook computers, virtual and/or augmented reality devices, high-definition televisions, digital video players and/or recorders, game consoles, satellite television receivers, wearable devices, personal digital assistants, mobile audio and/or video playback and/or recording devices, streaming devices, or any combination of the foregoing. Of course, subject matter is not limited in scope in these respects.

### Ballogy Curriculum

In embodiments, a curriculum (e.g., automatic assignments) aspect of an app may be designed and/or implemented as a way to develop athletes, for example. A curriculum may include fundamental drills utilized in years of coaching development teams, for example, and may divide them into various (e.g., three) skill-level categories, in embodiments. In embodiments, a curriculum may be fine-tuned by a team of experienced coaches and/or professional athletes for basketball, for example, and/or may be further developed and/or fine-tuned by sports experts who may analyze fundamental basketball skills and may design progressive drills to enhance them. An opportunity to scale this concept to multiple sports has been identified and experts have been gathered from respective fields to support the building of different curricula for various individual sports, for example.

In embodiments, an app may encourage athletes to work on their skillsets at home and may take into consideration that it may be advantageous for athletes to perform additional work outside of their organized practices and/or sessions to grow and progress in their crafts. In embodiments, a curriculum may target specified skills at least in part by incorporating a diverse range of drills and/or exercises focusing on shooting, dribbling, passing, defense, and/or overall athleticism, for example. Individual drills may be carefully selected to address key aspects of player development, in embodiments.

In embodiments, an app may provide a fifty-two week Workout schedule for athletes, for example. Workouts may change throughout the year to ensure progressive improvement, in implementations. In embodiments, a pre-season period may focus on skill-building and/or conditioning. Further, for example, an in-season period may emphasize game-specific drills. Also, an off-season period may be directed to skill refinement and/or overall development, in implementations. In embodiments, pre-season and/or off-season periods may also include additional drills and/or reps within a Workout, while in-season Workouts may be directed more towards maintenance and/or respect for a coach's time in incorporating game strategies and/or concepts, for example. In embodiments, an app, such as may be developed and/or provided by Ballogy, may offer solutions to fit a player's development in-season.

In implementations, a pre-season period may start off with drills, provided to a user via an app (e.g., a server computing device communicating with a player's remote computing device/mobile device), focusing on basic fundamentals such as defensive footwork and principles, ball-handling, shooting, finishing around the rim, etc. In embodiments, a pre-season period may begin with six drills within a Workout with an average of two or three repetitions per drill to get an athlete acclimated to player development prior to season start, for example. In embodiments, individual Workouts may be designed to take an athlete about 30-45 mins to complete. Within a pre-season, there may be ten Workouts corresponding to ten weeks of a typical pre-season period for basketball, for example.

In embodiments, in-season Workouts may comprise about two or three drills to fine-tune an athlete's ball-handling, shooting, finishing at the rim, footwork, etc., during a season, for example. Individual Workouts may be designed to last for about fifteen minutes strategically to either begin as a warm up or end practice with an exit activity, for example. Typically, a season is around twenty-two weeks in duration and therefore, in implementations, an app may provide twenty-two Workouts that may be randomized as a daily touch point for athletes, for example. In embodiments, during a season, coaches may focus on team strategies and/or concepts in a two to two-and-a-half hour practice setting, for example, and therefore Ballogy, for example, may provide a solution (e.g., via an app) to include individual player development. Of course, these are merely examples of the amounts and types of Workouts that may be selected, assigned, tracked, etc., via an app, and subject matter is not limited in scope in these respects.

In embodiments, offseason Workouts may comprise five or six drills, for example, that may start off as easing into targeted areas of refinement with a focus of improving an athlete's ball-handling, shooting, finishing at the rim, footwork, etc. Individual Workouts may be designed to last thirty to forty-five minutes and may have two phases. In embodiments, a first phase may begin with approximately three to five reps (i.e., repetitions) of each drill. In embodiments, halfway through an off-season, a level of intensity may rise in reps to challenge an athlete with a more rigorous workout, for example. In embodiments, an offseason may comprise twenty Workouts corresponding to twenty weeks for a duration of a basketball off-season in the spring and summer, for example. Again, these are merely examples of the amounts and types of Workouts that may be selected, assigned, tracked, etc., such as via an app, and subject matter is not limited in scope in these respects.

Additional discussion regarding Workouts, automatic assignment-type Workouts/curriculum Workouts, etc., that may be implemented, at least in part, via an app is provided below. For example, the discussion that follows may describe example aspects of “My Workouts,” “Team Training,” “Create Workout” (or “Curriculum”) and/or other functionalities of an application, such as an application that may execute, at least in part, on a mobile device, for example.

In implementations, several types of users may be supported. For example, user types may include coaches, players, and/or parents. “Team Training” aspects may be directed more particularly to coaches, for example. Also, for example, “My Workouts” aspects may be directed more particularly to individual users (e.g., athletes). In embodiments, a coach may join a group, for example, and/or may also create Workouts. Individual athletes may also create Workouts, join groups, etc. In embodiments, individual coaches may be provided, via an app, an access code to enter their group. Players (e.g., individual athletes) may be provided their own access codes to access group materials, again via an application executing on their respective devices, for example. Groups may also have owners (e.g., coaches, parents) and members (e.g., players, parents), for example.

One or more embodiments may comprise a “My Workouts” aspect of an app. In implementations, graphical user interface screens related to My Workouts may comprise a static screen for users (e.g., all users). In embodiments, a “Featured Workouts” aspect may be provided free of charge to users, although subject matter is not limited in scope in this respect. Also, in embodiments, Featured Workouts may be tagged as beginner, intermediate, and/or advanced. In embodiments, users may be provided, via an app executed on a mobile device, for example, Featured Workouts for their appropriate skill level (e.g., beginner, intermediate, advanced, etc.). In embodiments, a process for creating Workouts may be similar for coaches and individual players. In some implementations, Workouts created for individual users may not include beginning or end dates such as may be the case with coach-created Workouts, for example. In embodiments, self-assigned Workouts and group-assigned (e.g., assigned by a coach) Workouts may be displayed in a My Workouts aspects of an app.

In embodiments, a My Workouts aspect may be private for individual users. That is, for example, only a particular user may view or otherwise access the particular user's My Workouts content. In embodiments, if a user participates in public Featured Workouts, the particular user's progress and/or scores related to the Featured Workouts may not be private and may be viewed publicly.

In embodiments, creating a Workout for an individual from scratch via a My Workouts aspect may be free of charge, while creating a Workout from a template from a provided library may be accessible behind a paywall, for example. In embodiments, a monthly subscription plan may be implemented for some My Workouts features, such as, for example, creating Workouts from templates, viewing and/or otherwise accessing a Workouts Library, etc.

In embodiments, one Featured Workout may be selected per skill level. For example, users who select “beginner” as their skill level may see a beginner level Featured Workout. Intermediate and/or advanced players may see a Featured Workout appropriate for their skill levels, as well. As mentioned, Featured Workouts may be provided free of charge, in embodiments. A user may tap “View All Featured Workouts” to see whatever may be available for other skill levels, but the one displayed by default may be whatever matches their skill level.

In embodiments, a View Workout Library aspect may function like a simplified version of “Use an Existing Workout” within a Team Training flow, for example. Individual users “assign” Workouts from a library, via an app, to themselves, but without start/end dates, for example. In embodiments, as a user finds a Workout they like from the library, they can select it and have it added in real-time or near real-time to their “Active Workouts.” As mentioned, a Workout Library may be behind a subscription paywall, in implementations.

In embodiments, a My Active Workouts aspect may provide to a user a complete overview of everything the user has assigned to themselves and (if applicable) everything assigned to them by their coach(es) within any Team Training groups they may have joined. In embodiments, when a user has not joined any Team Training groups, we can assume that all Workouts are self-assigned, and team/user profile pics are not needed to designate the source of a Workout, for example.

In embodiments, Featured Workouts and Workouts assigned by a group may have an end date, and a “Complete this by . . . ” tag may be displayed above the title, for example. In embodiments, self-assigned Workouts may not have an end date, as mentioned. Rather, a “You started on . . . ” date may be displayed, for example.

In embodiments, particular Workouts, challenges, etc. may be sponsored. For example, a soft drink company may provide a month's work of soft drinks to an individual achieving a highest score for the month for a particular Workout. Of course, subject matter is not limited in scope in these respects. Sponsorship opportunities may also be provided in other aspects of an app. For example, advertising badges may be displayed for Workouts (e.g., such as those associated with a star player).

Also, in embodiments, a user may click on or otherwise select a “View Workout Rewards” link or the like and the user may, as a result, be directed to a Featured Workout Rewards display, for example. In this manner, a user may view, assess, and/or access rewards, such as legacy points and/or sponsored rewards, that may be achieved and/or that have been achieved by the user, in implementations.

In one or more embodiments, a “View Workout Library” icon is shown in gold, indicating that a particular user to whom this screen belongs is not subscribed to upgraded features and therefore does not have access to the Workout Library, in implementations. Conversely, the “Create your own Workout” icon is shown in red, indicating that the particular user has access to that particular feature, for example.

In embodiments, a user may click on or otherwise select a Featured Workout, and a My Workouts screen may be displayed, for example. In one or more embodiments, the left-most view shows a display prior to a user completing all or a part of the Featured Workout and the right-most view shows a display indicating progress towards completion of the Featured Workout, in implementations. Further, a user may provide inputs to an app to record completion of a drill, to provide a user's initials, to record reps completed, etc. In one or more embodiments, a user may be guided through various stages (e.g., three stages are depicted) of a Featured Workout, wherein the user may be directed to a next task upon completion of a prior task as part of the particular Featured Workout.

In embodiments, Featured Workouts may be changed, rotated, etc. once a week, for example. Also, in implementations, Featured Workouts may be displayed with a particular background color (e.g., black), while coach-assigned workouts may be displayed with a different background color (e.g., white).

In embodiments, if a user viewing a My Workouts aspect of an app is a coach and/or is otherwise an owner of a group (e.g., team or multiple players), the user may assign a Featured Workout to a group.

As previously indicated, a user may create their own Workout. In embodiments, a user may select “Create your own Workout.” In response, an app may be directed to a Create your own Workout flow, for example. In one or more embodiments, a user may select “View Workout Library.” As previously mentioned, in implementations, a user may be provided access to a Workout Library if subscribed. Responsive at least in part to a user selecting “View Workout Library,” an app may be directed to a Workout Library display. From this display, a user may click on or otherwise select a category and/or may view various Workouts within a selected category, for example. In embodiments, a user may preview a Workout by clicking on or otherwise selecting a particular Workout, for example. Once a user has created a Workout or selected a Workout from the Workout Library, the Workout may be viewed in an All Workouts display, for example. From this screen, a user may access the various Workouts and/or may be able to track progress with respect to the various Workouts. For example, a percentage may be displayed for each Workout indicating a percentage of assigned repetitions (reps) that the user has completed within each Workout.

In one or more embodiments, example Workouts (e.g., suggested practice routines) may be created from within an app, whereby a coach and/or an individual athlete may provide instructions or a routine to follow for a team and/or for individual players, for example. In embodiments, coaches, players, etc. may also upload video content that may or may not be connected with a particular Workout. In embodiments, existing Workouts may also be modified, via the app, by users (e.g., coaches, players, parents, etc.).

In implementations, a coach, for example, may select, via an application, any of a range of available pre-defined and/or existing Workouts. For example, Workouts related to shooting, finishing, dribbling, passing, rebounding, defense, conditioning, just for fun, mindset, etc. may be available via an app. In embodiments, a coach (or other group owner), for example, may select an icon via a graphical user interface of an app to view available Workouts for a selected category. In embodiments, Workouts may be categorized as beginner, intermediate, and/or advanced, and a group owner, such as a coach, may select an appropriate level of Workout to assign to individual players based, for example, on individual needs of a player.

In implementations, Workouts may be created and/or modified by coaches, for example. In embodiments, a coach or other group owner may select “Create a new Workout” or may select “Assign an existing Workout.” In the former case, the app may be directed to a create a new Workout flow, for example. In the case of a user selecting “Assign an existing Workout,” the app may be directed to an Assign an existing Workout flow, for example.

In embodiments, an application may provide an interface whereby the Workout creator/modifier may elect to keep the Workout private (e.g., available only specified individuals) or may elect to make the Workout available to others by selecting a “public” option, for example.

Further, for example, coaches (or other group owners, for example) may preview Workouts prior to assigning them. For example, a coach may view a portion of a Workout (Workouts may comprise video content) to assess its applicability and/or appropriateness before assigning a Workout to a player.

In embodiments, Workouts may include tasks for a player to perform and/or a coach may specify a number of repetitions for a player to perform, for example. A coach may also modify a sequence of activities for a player to perform, for example. In one or more embodiments, a number of customization options may be provided via an app, in embodiments. In implementations, a coach or other group owner may specify particular aspects of a Workout, but may be prevented from altering contents of a Workout. For example, if a Workout includes a shooting drill, a coach may specify a particular number of repetitions for a player to perform, and/or may specify a conditioning drill to be performed prior to a shooting drill, for example, but may be prevented from altering contents of the shooting drill itself.

In embodiments, once created, a Workout may be assigned, again via an app, to a single player, to a group, or to a subset of a group, for example. Start and end time and dates may be specified, for example.

Further, in embodiments, once assigned, a Workout may be viewed by a coach, for example, via an “All Workouts” screen of an app. This screen may show which Workouts are currently active and when they expire, for example. In embodiments, upcoming and/or archived Workouts may also be shown, and/or options for creating a new Workout or assigning additional existing Workouts may also be provided.

Additionally, in implementations, functionality may be provided by an app to automatically generate “Curriculum Workouts” (aka “automatic assignments”), for example. In embodiments, coaches may assign existing curriculum Workouts and/or may assign existing Workouts. These automatic assignment-type Workouts, selectable via an app, may provide a systematic and/or customized approach to training, for example. In embodiments, coaches may be assured that players following such curriculum Workouts will improve during the offseason, preseason, or in-season. That is, for example, curriculum Workouts may be automatically generated based on offseason needs for a player or group, for pre-season needs, and/or for in-season needs. In embodiments, start and/or end dates may be specified, such that a coach will not need to set new tasks, or create additional Workouts, or the like while the curriculum Workouts are active. Generally, players may be trained during these different phases of the year or season via their interaction with an app. That is, for example, an app may be thought of as training the players on behalf of a coach. Of course, a coach may design the type of program they might like their players to follow, but the details of the Workouts and assignment, as well as tracking, teaching, and reporting, for example, may be handled via a player's interaction with an app, in embodiments.

As an example, “NFHS Advanced” and/or “NFHS Youth Development” curriculum Workouts may be selected. Different curriculum Workouts may have particular shooting, finishing, dribbling, passing, rebounding, defense, conditioning, just for fun, and/or mindset, etc. Workouts as part of their particular plans for various phases of the year (e.g., preseason, offseason, in-season, etc.). Such automatic assignment-type Workouts may be designed by coaches or other experts to guide players through offseason development, preseason development, and/or in-season development, in embodiments. In embodiments, start and/or end dates may be pre-defined, so the coach wouldn't need to be concerned with that aspect. In one or more embodiments, curriculum Workouts may be assigned on a group basis, in some implementations. In embodiments, players in a group may be assigned these Workouts on behalf of a coach, for example, and/or coaches may select appropriate curriculum Workout for their particular groups.

In implementations, an application may designate a particular routine, such as a shooting routine, passing routine, dribbling routine, etc., or a combination thereof, as part of an automatic assignment-type Workout. In embodiments, a group that is assigned to a particular automatic assignment-type Workout may be assigned automatically assigned a particular routine. For example, an application, perhaps being executed at a server computing device, may push an indication of this new assignment to members of groups that have been assigned (e.g., by their coaches or other owners) a particular assignment-type Workout. In other words, rather than a coach going through the process of selecting and/or creating Workouts described previously, such assignments may be made automatically in accordance with a selected curriculum Workout managed automatically via an app, in embodiments.

In embodiments, once a new routine/Workout has been assigned as part of a curriculum/automatic assignment-type Workout, individual players belonging to groups subscribing to the particular curriculum/automatic assignment-type Workout may receive a message, such as via a pop-op alert on their individual electronic devices, and individual players may then access the new routine/Workout via the “All Workouts” screen of an app, for example.

In embodiments, responsive to a user, such as a player, for example, selecting a particular Workout via an app, the app may display to the user a page that may communicate various aspects of the assigned Workout. For example, the specific drills to be completed, the number of reps to be completed, etc. may be displayed. Further, an app may track, such as via video input as the user performs the assigned tasks, the user's progress in completing aspects of the assigned Workout.

In embodiments, an app may direct a user to a screen showing information regarding tasks to be completed. For example, an app may display video content to the player (or other type of user) via the user's electronic device (e.g., tablet, cell phone, etc.) to demonstrate to the player how to execute the task. For example, responsive at least in part to a user selecting a particular element of a particular screen in an app, the app being executed on the player's electronic device may communicate with a server computing device, for example, and video content may be communicated to the user's electronic device and displayed to the user. Various communication, server, and/or network technologies are described herein.

Additionally, in embodiments, an app being executed on the player's electronic device may provide a user interface element whereby the player may initiate a video recording. In embodiments, a video recording may be made via an app to record the player executing the assigned task. In embodiments, recorded video content may be analyzed in real-time or near real-time via AI and/or other machine-learning techniques, for example, and/or may be provided to remote computing devices to enable analysis by other devices or individuals (e.g., coaches). Also, in embodiments, a coach may designate a particular Workout as “non-video” or the like to indicate that the player is not asked to provide a video recording. In implementations, an app may grey out the recording feature of the user interface in such circumstances. Alternatively, for a no-video task, an app may display to a player a means for the player to manually enter the player's progress (e.g., “completed” and/or the like). If it's a scored task, the player may add or edit their score via the user interface of an app, for example.

In implementations, once a player, for example, uploads a video, the video may become available via an app on a player's “individual results” screen. Also, for example, an individual results screen may associate a score, such as may be generated by an app using AI/machine-learning analysis (e.g., including communication between a user's electronic device and a server computing platform) and/or by a coach's analysis, with an uploaded video. A coach, for example, may analyze a player's uploaded video and may determine a score to assign to the uploaded video. That is, in embodiments, a coach may analyze a player's performance of the assigned Workout using the player's uploaded video. As mentioned, such analysis may be performed by AI/machine-learning analysis in some implementations.

Also, in embodiments, an app may provide instructions to a coach on how to score particular Workouts. In this manner, scoring from one coach to another may be standardized to a large extent. In embodiments, some Workouts may not involve scored Workouts, but instead may be designated as non-scored. In this circumstance, if it is a video-recorded Workout, a coach may simply indicate that a particular Workout has been completed, as evidence by the player's uploaded video, for example. As mentioned, some Workouts may ask the player to self-report. In the case of a coach-scored Workout, the player may receive a notification, via an app, that a coach has scored the Workout, for example.

In implementations, feedback may be provided to the player for completed Workouts. An app may provide a chat feature whereby a player and coach may communicate with each other, for example.

As mentioned, for some Workouts a player may be asked to provide a score for a completed task. In one or more embodiments, an app may display to the player guidelines for scoring an assigned Workout. For a training task, for example, a player may tap a “mark as completed” button on a graphical user interface (screen) and/or may be asked to enter a player's initials via a graphical user interface, for example. In embodiments, for scored Workouts, a player may self-assess in accordance with provided guidelines or instructions provided by an app and the player may enter a score. As also mentioned, Workout analysis and/or scored task analysis and/or scoring may be accomplished via AI/machine-learning implementations, which may include communication between a server computing device and a user's device, for example.

As mentioned, coaches or other users may assign Workouts to players or groups of players, for example. In embodiments, a coach, may for example, design their own training program for one or more players by selecting pre-existing Workouts to assign to a group or individual. As also mentioned, curriculum Workouts (automatically-assigned Workouts) may be selected by a coach to assign to groups or individuals. With curriculum-type Workouts, a coach may design their own based on existing Workout routines in the various available categories (shooting, passing, defense, conditioning, etc.) and may manually manage the assigned Workouts throughout the course of a cycle (e.g., off-season, preseason, in-season). A coach may also assign app-generated and/or managed curriculum to individuals or groups, for example. In this case, an app may automatically assign particular Workouts in the various available categories as appropriate given the cyclical nature of the basketball sporting calendar, for example (of course, subject matter discussed herein may be expanded to include other sports or skilled activities). For example, an app may assign particular Workouts in the offseason, may alter assigned Workouts as the offseason progresses, may again alter assigned Workouts in the preseason phase, and/or may further adjust assigned Workouts during the in-season phase. In implementations, a coach need not manually manage these aspects for curriculum-type Workouts.

Also, for automated curriculum-type Workouts (e.g., offseason, preseason, in-season cycle), assigned Workouts may be adjusted based on individual player's progress and/or development as determined either by a coach and/or as determined via AI/machine-learning analysis, in embodiments. In embodiments, both a coach and a player may monitor the player's progress, whether that progress be evaluated by a human coach or whether evaluated via AI/machine learning.

As an example, basketball tends to be cyclical and generally fixed in terms of off-season, preseason, and/or in-season. An app may be implemented to understand that from late August/early September to the end of October may comprise a preseason and that October going into November is generally when the basketball season starts for every single high school amateur team across the country in the United States. The offseason may be considered to start in April and may continue until the start of the preseason, for example. An app may be programmed with particular start and/or end dates for the various phases of a sporting calendar. Also, an app may also track start and end dates for the various cyclical stages of a calendar for a given sport for any number of different schools, leagues, organizations, etc.

In embodiments, an app may automatically assign Workouts, tasks, etc. based on this cycle or coaches may assign Workouts as they see fit. For example, an app may generate a particular Workout curriculum for the preseason phase. A coach may select particular Workouts from that curriculum to assign to players, or a coach may assign an automated curriculum (automatically assigned Workouts) which may be managed by an app, wherein an app automatically assigns particular Workouts, tasks, etc., depending on whether it's the offseason, preseason, in-season, for example. Workout plans may also automatically be altered during a particular phase of the calendar year, in some implementations. In embodiments, different Workouts may be automatically assigned on particular days depending on a team's specific schedule, for example, or a coach may assign particular tasks, Workouts, etc. depending on a coach's team's schedule. A coach may also opt-in to individual Workouts and/or a Workout plan as they deem it advantageous for individual players and/or groups (e.g., teams), for example.

In implementations, a Workout plan, such as an automated curriculum plan and/or a coach-generated plan, may include start and/or end dates for when particular Workouts “go live.” An app may monitor time and date and may automatically assign a particular Workout to appropriate player(s) when a specified time and/or date is reached. For example, an app or a coach may designate a particular Workout to be assigned to a group at a particular time and/or date. When that time and/or date arrives, an app automatically assigns the Workout and the Workout may be visible to the player(s) via an app (e.g., an app or portion of an app being executed on their personal electronic devices, such as a cell phone, tablet, etc.). The player may be notified via a pop-up display on the player's electronic device, for example.

Restating some of the above, in embodiments, an app may automatically generate a Workout plan (“curriculum” plan) that may include a number of Workouts from various categories, for example. In embodiments, automatically generated plans may change during the course of a sporting cycle, such as basketball's general offseason, preseason, in-season cycle, for example. Various Workouts included in a curriculum plan may be automatically assigned to players at appropriate times and/or dates. Also, for example, coaches may select a subset of curriculum plan Workouts to assign to individuals or groups, or may add Workouts to a curriculum plan. Coaches may also design their own curriculum plans, for example. One advantageous aspect of an app (e.g., the “Ballogy” app) is that many of the arduous tasks involved in coaching may be transferred from a coach to the app, and may help more efficiently and/or effectively develop players and/or teams. AI/machine-learning analysis may add to these advantages by helping coaches evaluate players, Workouts, tasks, drills, etc. and/or by providing timely feedback to players as they work to perfect their techniques and skills. An app may also help organize a coach's efforts to personally guide players and teams in their development, for example.

As mentioned, in some implementations, an AI/machine-learning analysis engine may analyze a player's uploaded video content, for example, to score a task or Workout and/or to analyze a player's technique, movement, skill completion, etc. In implementations, an app may also include a recommendation engine that may, based at least in part on AI/machine-learning analysis, recommend to a coach, player, parent, etc., particular Workouts to be assigned to the player to develop the player's performance. Such recommendations may be customized for individual players based. Also, a recommendation engine may rely not on AI/machine-learning analysis of video content, for example, but may instead, or in addition, base the recommendations on a player's results for a particular assessment, Workout, task, etc.

For example, a Ballogy Skills Assessment (BSA) may measure a player's ability to make a specified number of basketball shots from specified areas of the basketball court in a specified order in a specified period of time, as explained previously. A recommendation engine may obtain results of such an assessment, with or without accompanying video, and may recommend particular Workouts for the player based on the assessment results. For example, if a BSA shows a particular player missing a relatively larger number of shots in a particular region of the basketball floor (e.g., 3-pt line, right left wing), an app may recommend Workouts designed to improve a player's skill in making shots from that region of the floor. As mentioned, recommendations may also be based, at least in part, on AI/machine-learning analysis of a player's techniques, skills, movement, etc., as evidenced by recorded video content, for example.

In implementations, recommendations may be suggested to a player and/or a coach, for example, via a “My Ballogy” screen portion of an app. A player may be provided, via an app's graphical user interface, an ability to “tap” on a particular zone of the basketball floor to prompt recommendations regarding Workouts. For example, a particular zone of the basketball floor may appear “red” after a shooting assessment, indicating a larger number of missed shots for that zone. The player may wish to be assigned Workouts designed to improve shot-making from that zone, so the player may select, as via a tap to the graphical user interface, the zone in question. The recommendation engine may then determine one or more appropriate Workouts for the player, and the recommended Workouts may be displayed to the player and/or an option to select one or more of the recommended Workouts may be provided by an app. Similarly, coaches may be provided options to assign Workouts based on a player's skills assessment, in some implementations.

In embodiments, a player may elect to engage in a “free shoot” whereby the player simply takes shots from wherever they want on the basketball floor. Video analysis of the shooting session, such as via AI/machine-learning, may track makes and misses and may track where on the floor the shots occurred, in implementations. In embodiments, recommendations may be provided for this type of assessment as well, based at least in part on the analysis of hits and misses.

In some implementations, such as where video analysis may not be available and/or where AI/machine-learning is not available, recommendations may be made based at least in part on multiple results of skills assessment sessions. For example, if the aforementioned BSA is performed multiple times by a particular player and the results are recorded in an app (e.g., even if self-reported rather than via video analysis with or without AI/machine-learning), recommendations for Workouts from the curriculum may be generated and communicated to the player, coach, parent, etc., as appropriate, by an app. Again, such recommendations may include communication between a user's electronic device (e.g., cell phone, tablet, notebook computer, etc.) and a remote computing device (e.g., server computing platform) by way of one or more communication techniques discussed herein.

In other implementations, a recommendation engine may design its own Workout, such as a customized shooting Workout, based on AI/machine-learning analysis of a player's shooting session (e.g., free shoot or structured routine, such as BSA), rather than assigning a pre-existing Workout from an app's catalog/curriculum.

As previously mentioned, coaches may design Workouts and may select whether to make the Workouts public or to keep them private (e.g., available to only specified individuals and/or groups), for example. In embodiments, a recommendation engine may add such public-specified coach-designed Workouts to the pool of available Workouts that may be recommended.

Also, in embodiments, a coach or other Workout designer may be provided, via an app, an ability to not only indicate public or private status, but may also indicate one or more categories (e.g., shooting, dribbling, passing, etc.) that appropriately categorize their drill. A coach or other Workout designer may also be provided a graphical user interface element by which a coach or other Workout designer may indicate one or more particular zones of a shot chart, for example, that may correspond to the new Workout. For example, Workouts aimed at improving finishing around the rim might be categorized differently than Workouts directed at improving 3-point shots from the corner, and a coach or other Workout designer may indicate such via a graphical user interface element, such as a displayed shot chart, for example. This feature may also be implemented in connection with other aspects of an app, such as the BSA, for example. And, of course, such functionality is not limited to shooting drills. Dribbling, passing, and/or finishing drills, for example, may take place in various zones of a basketball court, and coaches or other Workout designers may indicate such zones via an app's graphical user interface, such as during the “create a Workout” process, for example. Further, a recommendation engine may consider such “zone” information when recommending particular skills, drills, Workouts, etc. to particular players and/or groups.

A need has been established to determine the shooting capabilities of a basketball player, for example. To evaluate a player's abilities, it may be advantageous to focus on the most frequently used shots in the modern game. Ballogy Skills Assessment (BSA), for example, may showcase a player's ability to make shots from close up, mid-range, and/or three-point distances, for example, to allow the player to display their ability from different spots on the floor. It may be advantageous for such an assessment to apply to all levels of players to give everyone a chance to see where and/or how they measure up. A number of variations of the tests have been utilized, and a 40-shot test may be advantageous because it may be accomplished in 4-minute increments, for example.

In embodiments, BSA may be advantageous because a disparity in training and evaluating talent in the amateur basketball space has been observed. A desire for skills assessment that could easily give a player, coach, or scout the ability to quickly tell what skill level a player is at has been identified. In embodiments, BSA (Ballogy Skills Assessment) may evaluate a player's shooting ability and/or may easily and/or readily compare the player's skill with their peers. There has been, historically, a missing “standardized” element to tracking and measuring skills development because, for example, everyone trains in their own way. For the player, the BSA gives them a chance to showcase their skills to the world.

It may be noted that BSA may be implemented, at least in part, via one or more computing devices, processing circuitry, sensors, communications technologies, etc. For example, BSA may comprise, at least in part, a software application that may be executed by any of a wide range of electronic device types, such as those mentioned and/or discussed herein, including, to name but a few examples, IoT-type devices, tablet devices, cellular phones, notebook computers, desktop computers, etc.

Ballogy Skills Assessment may comprise the first objective skills metric that gives amateur athletes everywhere a chance to get noticed at the middle school, high school, and collegiate levels. Ballogy Skills Assessment is the first shooting test publicly accepted and endorsed by all levels of basketball by the NFHS, NHSBCA, NAIA, and NJCAA coaches alike, for example.

In embodiments, Ballogy Skills Assessment may be implemented in an app (e.g., software and/or firmware agent) that may be executed, at least in part, on a player's electronic device, such as IoT-type device 200, for example. Content related to the BSA may be provided to the player's device by a software and/or firmware agent executed at an administrator computing device, such as server 120, for example. Collectively, software and/or firmware agents executed, at least in part, at one or more user devices (e.g., IoT-type devices associated with one or more players, coaches, parents, etc.) and/or at one or more administrator-type devices, such as servers 120, may be referred to herein as an “application” or “app.” Various functionalities and/or capabilities may be partitioned among the various devices, in some implementations. In other implementations, individual copies of an app and/or different versions of an app may be executed at different devices. For example, a first implementation of an app may be provided to mobile devices associated with players, coaches, etc., and/or a separate implementation may be executed at one or more servers, for example.

In embodiments, a user may share BSA content via social media. For example, an app may generate a video clip (e.g., fifteen seconds) depicting a portion of a player's BSA, and the player may share that video clip via social media. Also, for example, a player may share a BSA score via social media, for example.

One may ask why does an app focus on certain shots, order, and length? In embodiments, an app may focus, at least in part, on close-up finishes because that may give a player the ability to showcase their athleticism or lack thereof. In embodiments, athletic players have the opportunity to dunk the ball and/or be creative on their first two shots in the BSA. It also emulates the ramp-up time before a basketball game and emphasizes that the game should be played from the inside out. In embodiments, BSA may be designed that way to give the player two easy shots to get off to a positive start to the test. In embodiments, each lay-up may be counted as one point because of the level of difficulty.

Moving on to the mid-range, mid-range shots off of the dribble are designed into the test to incorporate shooting quickly while dribbling, for example. The mid-range shot is statistically the next highest-made shot but it is quickly dying in the modern game of basketball so an app may specify a mere ten mid-range shots in the BSA, for example. In embodiments, this shot type may be included because it shows how quickly a player can explode into their dribble pull-up showcasing their efficiency of movement. Each mid-range pull-up jump shot is counted as two points because of the level of difficulty.

Moving onto the three-point shots in the BSA. The “3” is the most commonly shot in the modern game so we made the bulk of the test. In embodiments, BSA may incorporate shots on the move and stationary shots to assess the player's shot-making abilities in different common game-like scenarios. The catch-and-shoot three is the most common shot taken in modern basketball. In embodiments, an app mixes in a not commonly used shot which is a shot-fake three-pointer, mimicking a game-like scenario where if a player has hit two shots from one location the defense is most likely to try to stop that third shot then the offensive player will need to use a fake to relocate to a new location to shoot the ball. This is an advanced-level shot but it allows evaluators (or AI/machine-learning analysis) to quickly assess a player's skill-set. In embodiments, an app may instruct a player to continue to take a catch and shoot three-pointers but with a conditioning element to it. This may emulate a game-like situation where a player may sprint to a new spot and get their body and feet properly set to get a quality shot off. Each three-shot is counted as three points because of the level of difficulty, for example.

In embodiments, finishing a BSA with 3 free throws may be advantageous because basketball games may be won and lost at the free-throw line. A player's heart rate is usually pretty high at this point in the assessment and forcing them to slow their breathing and make free throws closely mimics a game-like scenario where they need to make free-throws from running up and down the floor a few times.

One may ask what might be an advantageous period of time for a BSA. In an implementation, the BSA may have a specified duration of four minutes (this is just an example—other lengths are possible in various implementations) because the average high school basketball gym has six baskets and if properly ran, BSA may involve three players which may allow an average basketball team including all twelve players to complete the Ballogy Skills Assessment as implemented in an app in less than fifteen minutes. It may be a factor for universal adoption by coaches to incorporate the test during practice time.

In embodiments, BSA, via an app, may represent a basketball player's first time truly testing their shooting ability with specified time and/or instructions. This may allow a player to test their ability to apply instructions without overthinking, focus on shot-making, and/or quickly get over potential missed shots. In embodiments, after a player receives their first baseline score, they have an indicator of where their shot-making abilities stack up with the rest of the Ballogy community and will know where the deficiencies in their offensive game. For example, if they do not make a high percentage of their three-point shots they know to practice that shot more often. This type of data-driven feedback is a factor to improving a player's skills in sports.

In embodiments, some example (e.g., key) factors measured in BSA may include:

Speed may be measured in multiple (e.g., two) ways. From watching the player move within the test, the naked eye (or, for example, AI/machine-learning video analysis) can see if the player is moving quickly from spot to spot. In embodiments, a timed element of the test also easily determines how quickly the player completes the assessment.

Pressure may be measured because there is the timed and recording element. Basic psychology shows that when someone is being recorded doing something it brings a level of focus and pressure to the performed action. That was part of the thought process but an objective was to determine the quality of the assessment we needed to ensure that each shot was captured by a live video recording device.

In embodiments, comprehension may be measured by the ability to understand and apply the instructions to the test. For example, if a shot is not completed in the proper sequence, then an app may disqualify that particular shot and shots to follow.

In embodiments, emotionally stability may be measured by not forcing the player to make a shot to move on to the next shot. In implementations, BSA may be about following the test and attempting every shot instead of having to make the shot move to the next one, for example. Many players may easily become frustrated and lose their emotional stability throwing off the rest of their BSA submission. Those who can remain stable and focus on one shot at a time usually excel at Ballogy Skills Assessment, for example.

In implementations, BSA may be scored from 0-100. In embodiments, scores range from: 90 and above is an excellent score; 70-89 is above average score; 50-69 is an average score; 49-30 is below average score; 29-0 is a poor score. An app may chart the progression of a player's BSA score from graphs and group them in leaderboards within an app as well. The type of feedback that may be provided to each athlete is feedback on how to improve their BSA score not on shot technique or form, although that may be implemented as well. An app may also provide feedback on how to apply the instructions better to improve a player's chances of scoring higher, for example.

How the Ballogy Skills assessment may be broken down set by set.

### SET 1: Lay-Ups (2 Shots)-Max Points=2

Starting on the right wing of a basketball court, drive right to attempt a right-hand lay-up. Next, the player may be instructed to run to the left wing, catch the ball, drive left, and attempt a left-hand lay-up. The assessment may begin simply, with a layup from the right wing followed by a layup from the left wing. These two shots may be thought of not only as a warmup but as part of a demo reel for a player. A player may be as creative as they want, with reverse layups, euros, dunks . . . whatever showcases their skills the best. Each shot is worth 1 point, for example.

### SET 2: One Dribble Pull-Up (10 Shots)-Max Points=20

Starting in the right corner behind the three-point line, a player may be instructed to attempt a 1-dribble pull-up jumper going to the left. This may be repeated four more times, for example, from the right wing, top, left wing, and/or left corner. Next, the player may do the reverse-one dribble pullup going to the right, from the left corner, left-wing, top, right-wing, and then in the right corner where the player began. Each of these 10 shots is worth 2 points.

### SET 3: Catch and Shoot and Flyby 3-Pointers (15 Shots)-Max Points=45

From each of the five spots (beginning in the right corner and advancing to the right wing, top, left-wing, and left corner), the player may attempt the same 3-shot sequence. The first two shots from each location are simple catch-and-shoot jumpers. The third shot is a pump-fake, side-dribble in either direction (your choice), aka a “fly-by.” This is a game-situation shot where your defender bites on your fake and you relocate to get off an uncontested shot. Each of these fifteen shots is worth three points.

### SET 4: Star Shooting 3-Pointers (10 Shots)-Max Points=30

Beginning in the left corner, a player may attempt catch-and-shoot 3-pointers from locations that draw the shape of a star. From the left corner to the right wing, left-wing, right corner, and top, these five shots create a first “star.” Sprint back to the left corner for five more shots in the same star formation to complete this ten-shot set. Each of these ten shots is worth three points.

### SET 5: Free Throws (3 Free Throws)-Max Points=3

At every level, games are won and lost at the free-throw line. Finish the BSA strong by making 3 free throws, worth 1 point each.

BSA—how it is computed and applied/used, for basketball and other sports (and, potentially, outside of sports).

Each user profile has a BSA “credit” by default when they sign up for an app. Additional credits can be purchased. Each BSA video submission uploaded uses one credit, regardless of whether the video is considered valid or not.

Before starting the BSA, the user can choose to record and submit for themselves (to add the resulting score to their own profile) or record on behalf of someone else. Choosing this second route requires either an email or phone number where a unique code will be sent after the BSA recording is completed and uploaded.

In implementations, if the user uploads their own profile, they receive a message stating that they'll receive their score and Legacy Points after their video is reviewed (e.g., by a human and/or via AI/machine-learning analysis). The video may be uploaded to Vimeo, for example, and then appears in our browser-based Admin Panel (and aspect of an app, such as may be executed at a computing device) where a Ballogy staffer can watch and manually denote makes and misses for each shot. In other implementations, analysis may be performed in an automated fashion via machine learning/AI. This process simultaneously time-stamps the video so that when it ultimately gets displayed in an app, viewers can tap on individual shots and automatically jump to that portion of the video. Once all shots are designated as make or miss, a score is produced and sent to the user, and the score/video appears in an app-wide BSA leaderboards.

If this video was recorded on behalf of someone, a BSA Score Code should be sent to either the email or phone number provided; whichever user profile syncs this code will have the resulting BSA score/video associated with their profile. In implementations, the score/video may not appear on BSA leaderboards until the code has been synced. If the BSA video submission failed to meet all of the requirements, as judged by the Ballogy staffer reviewing it and/or via AI/machine-learning analysis, it is deemed “invalid.” A notification is returned to the person who submitted the video letting them know that their submission was invalidated.

In implementations, video analysis may be performed by and/or assisted by artificial intelligence/machine learning aspects of an app.

In implementations, each user profile has a BSA “credit” by default when they sign up for an app. Additional credits can be purchased. Each BSA video submission uploaded uses one credit, regardless of whether the video is considered valid or not.

Before starting the BSA, the user can choose to record and submit for themselves (to add the resulting score to their own profile) or record on behalf of someone else. Choosing this second route may require either an email or phone number where a unique code will be sent after the BSA recording is completed and uploaded. The user opens a BSA session which triggers the computer vision (e.g., AI analysis, machine-learning, etc.). The user recording the video will see a real-time score update as each shot is attempted. Each shot is designated as either a make or a miss from the computer vision.

At the end of their 40 shots (or time expiration), they should already see their score. It is up to them to decide whether or not they want to upload their video. If they do, all of the same mechanics apply as before, except that Ballogy staff can review videos to modify or invalidate inaccuracies in Vision (e.g., AI/machine-learning analysis), but doesn't need to for scores to be populated on an app. Immediately after uploading, they should receive their Legacy Points (equivalent to their BSA score+100 additional points). If the score was for someone else, they should immediately receive their code to sync. That score will sync with their profile and remain there.

In embodiments, Ballogy Pre-Camp Assessment may be based on Dribbling, Layups/Finishing, and Shooting Fundamental Skills. In implementations, there is a goal range requirement for each Skill. Dependent on the outcome of the given skill assessment will determine the score for each Fundamental Skill, for example.

In embodiments, Ballogy Scores for each Fundamental Skill assessment are between 1-3 with 1=Beginner, 2=Intermediate, 3-Advanced. After completion of the 3 assessments, the average of the 3 scores will be taken determining the overall BALLOGY SCORE and grouping of the athlete based on skill level, for example.

In implementations, the average score among the three categories may be a deciding factor of where to place the camper (e.g., player).

In embodiments, a Ballogy Score may comprise a benchmark for beginner, intermediate, and advanced skill levels allowing coaches to evaluate their athletes before camp or tryout settings. It was created for various reasons, including customized learning experience. A pre-camp assessment tool allows camp organizers to gain valuable insights into each camper's current skill level, in implementations. This information enables camp staff to tailor the camp experience to meet the unique needs of every participant, ensuring that both beginners and advanced players receive instruction that is appropriate for their abilities.

Efficient Resource Allocation. In embodiments, coaches may be assigned to each skill level. By assessing the participants' skills in advance, camp organizers may better allocate resources. For instance, they can assign coaches or instructors with the right expertise to work with specific skill levels. This ensures that resources are utilized efficiently, leading to a more effective camp experience, for example. Enhanced Camper Engagement. Leveling the playing field. Campers who feel that their individual goals and skill levels are acknowledged are likelier to be engaged and motivated throughout the camp. In embodiments, a pre-camp assessment tool may foster a sense of ownership and/or personal investment in the camp experience. Streamlined Camp Planning: In embodiments, a tool may simplify camp planning by providing data on camper skill levels and grouping. It may be considered the standard assessment for camps due to its comprehensive nature, providing valuable insights into athletes' strengths, weaknesses, and areas for improvement, thus enhancing the camp experience. The assessment measures important aspects of the game such as dribbling, finishing, and shooting, in embodiments.

In embodiments, an app may focus on particular specified shots. For example, an X-Out Layup drill showcases the ability of an athlete to finish at the rim within a short amount of time. In implementations, a Hot-shot drill is used as a shooting assessment as it highlights the ability to make consistent shots at various areas on the court. In embodiments, a specified order of assessment drills may be designed to progressively challenge athletes. For example, a specified order may include starting with dribbling skills, moving on to finishing and gradually advancing to more challenging drills like hot shots that require movement around the court, shooting under pressure, accuracy, and consistency. The length of time of an assessment may be structured to cover a comprehensive range of basketball skills within a reasonable timeframe, ensuring thorough evaluation while optimizing efficiency. Essentially, it may be advantageous to not lose the attention the athlete and to therefore create an assessment that could be completed in under 5 minutes, in embodiments.

Embodiments may include a Ballogy Recommendation Engine, providing separate functionalities for players and coaches for basketball and/or other sports (and, potentially, outside of sports).

### Ballogy Recommendation Engine for Players

Shoot-Around Session: A user can open an app and launch a “shoot-around” session and shoot from any location on the court at random. In embodiments, vision technology (e.g., AI/machine learning analysis implemented in an app) gives real-time data on make, miss, and location via an app. After the session is stopped, the user gets a report that calculates, makes, misses, shooting percentages from each shot location and overall shooting percentage, for example.

Option A: Generative AI may suggest areas for improvement based on shooting percentages from quadrants on a shot chart. In embodiments, a player may ask the AI questions on how to get better at a particular skill in a search engine format. For example: “How can I improve my left-hand layups?”

In embodiments, a Vision (e.g., AI/machine-learning analysis) Shoot-Around Feature may include: An app gives real-time data on: Make, Miss, Location. After the “session” is done, an app may provide the user a report on makes and misses, for example. A sharable shot chart with shooting percent from each location may be generated. In embodiments, a player can input their geo-location (gym, school, or park). In embodiments, video may be accessible via a player's profile on an app. In embodiments, may work indoors at half-court. In implementations, without Wi-Fi access, video may be saved to the player's phone.

### Ballogy's Recommendation Engine for Coaches

Application 1: A coach may have an ability to upload game tape to a desktop version of a Ballogy app. From a vision/computer aspect, an algorithm, implemented in an app at least in part, may analyze game tape and/or may suggest Ballogy tasks/Workouts for a coach to assign to their team and/or individuals via a Ballogy mobile app. For example, if a team produces a large number of turnovers, a vision aspect of an app may communicate with an AI algorithm and/or may suggest to a coach to assign a variation of ball-handling and/or passing drills.

Application 2: A coach may have the ability in a group to “ask” a generative AI aspect to create or suggest a Workout for a team or individual(s). In embodiments, generative AI may source public workouts and/or tasks to create a unique Workout for a coach to assign to their team. In embodiments, a Ballogy app may tag individual tasks and/or Workout that are created properly.

Application 3: A coach may instruct an AI aspect to analyze a video of a recorded task and/or Workout and to generate feedback for a team or individual player(s).

As discussed previously, embodiments may include a sports social media application (e.g., “Ballogy app”) that may utilize machine/computer vision features and/or operations, for example, to determine if a player makes a basket, hits a pitch, throws a strike, etc. For basketball, for example, a computer vision aspect of the sports social media application may provide shot detection capability for players, coaches, teams, groups, etc. Also, for example, individual players may utilize computer vision aspects of the application to track shots (e.g., lay ups, free throws, mid-range jump shots, three point shots, etc.) over time, for example.

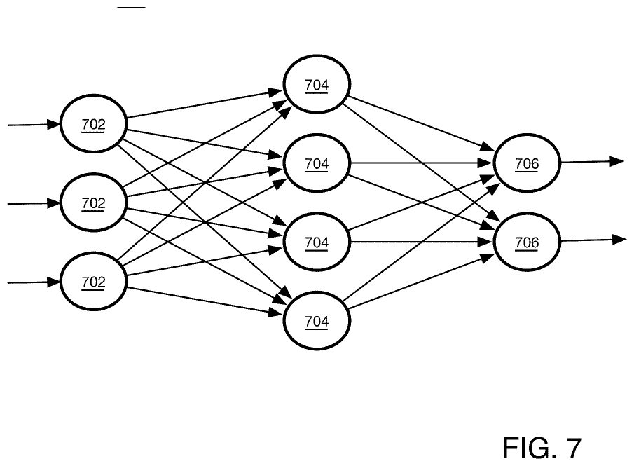

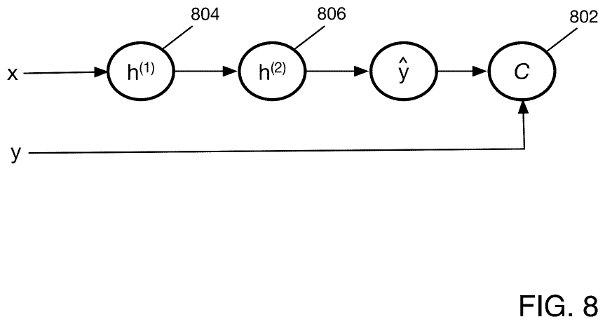

As also discussed previously, a computer vision aspect of a sports social media application may be based, at least in part, on AI/machine learning. In one or more embodiments, AI and/or machine-learning aspects may be implemented, at least in part, in hardware, software, or firmware, or any combination thereof. Embodiments pertaining to AI/machine-learning are discussed more fully below in connection with FIGS. 7 and 8, for example.

In one or more embodiments, a sports social media application may capture video content via a camera, such as via a cellular telephone or other electronic device. Also, in one or more embodiments, video content may be uploaded to an application by a user (e.g., application administrator, player, coach, group owner, parent, sports organization official, etc.), for example. In one or more embodiments, an application may perform machine vision (e.g., AI, machine-learning, etc.) analysis on such video, thereby generating test performance data, shooting session statistics and/or results, etc.

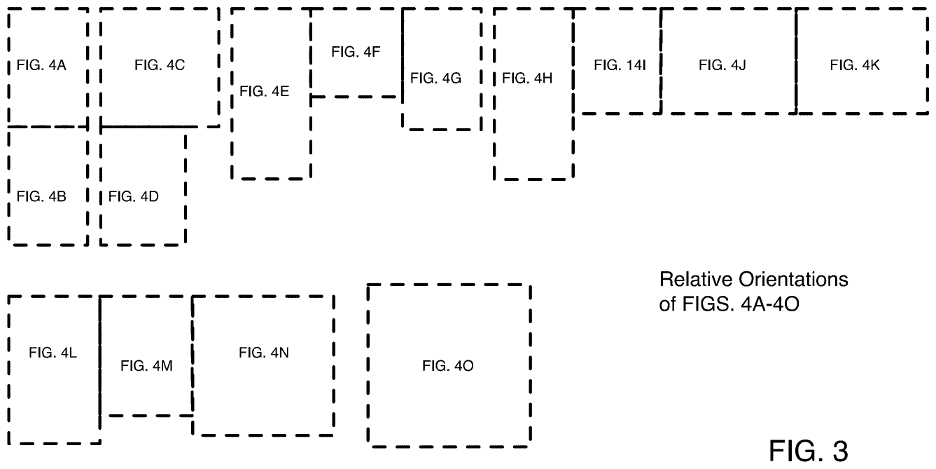

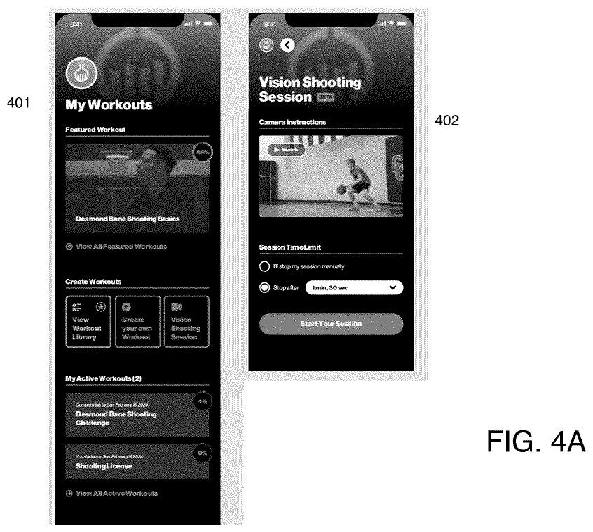

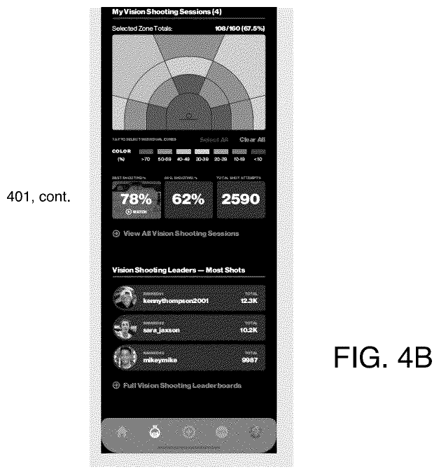

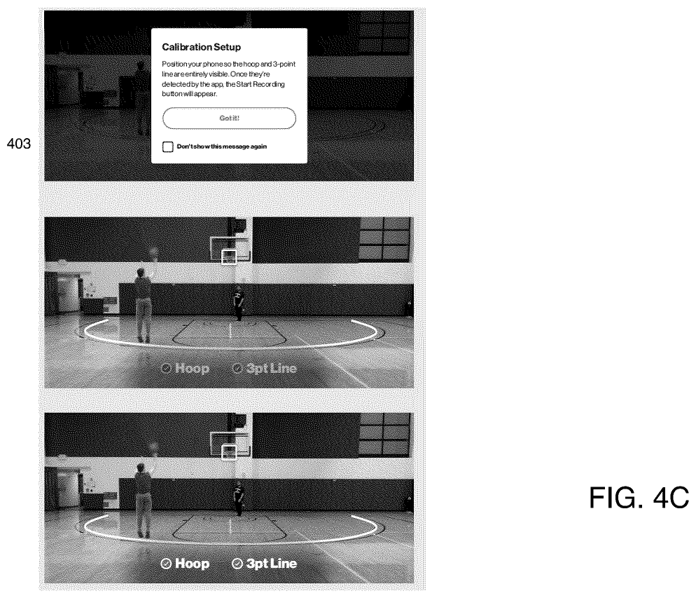

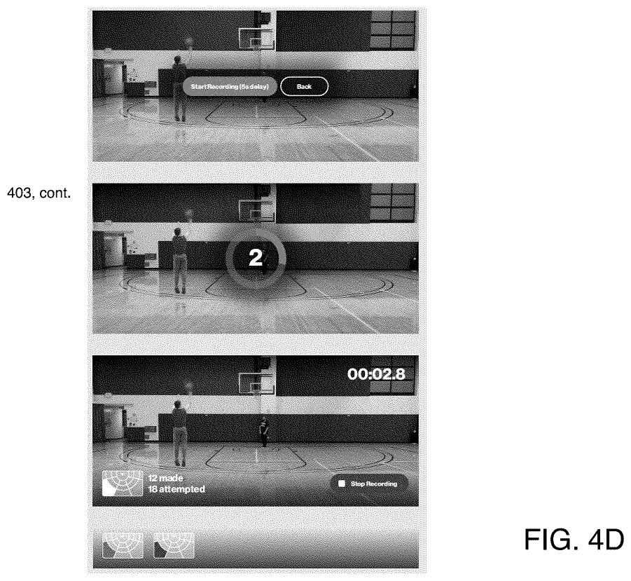

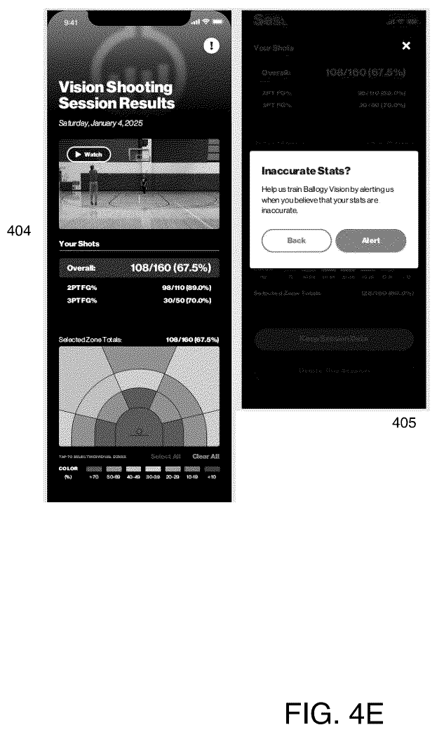

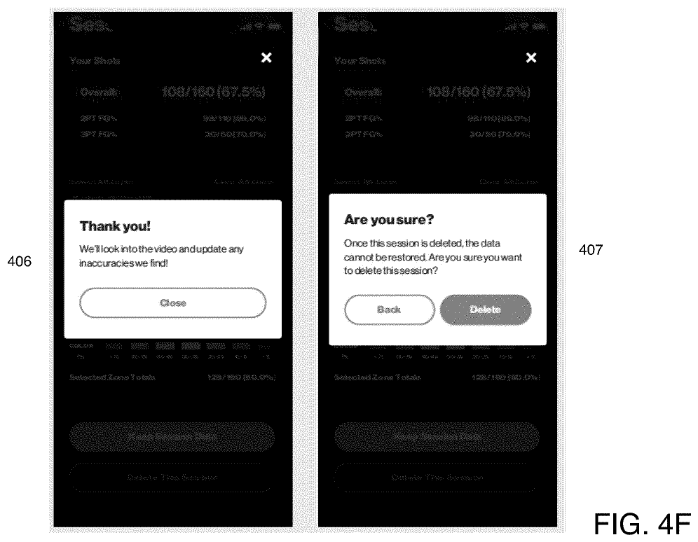

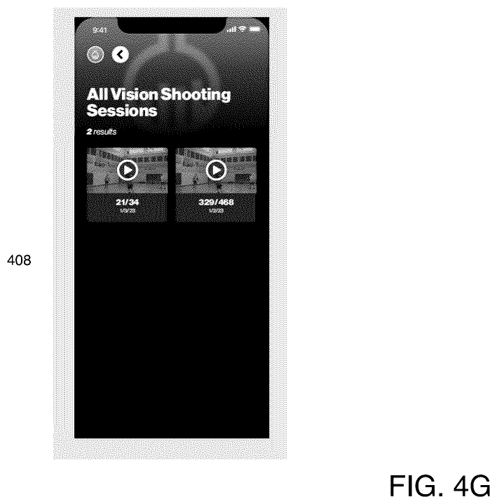

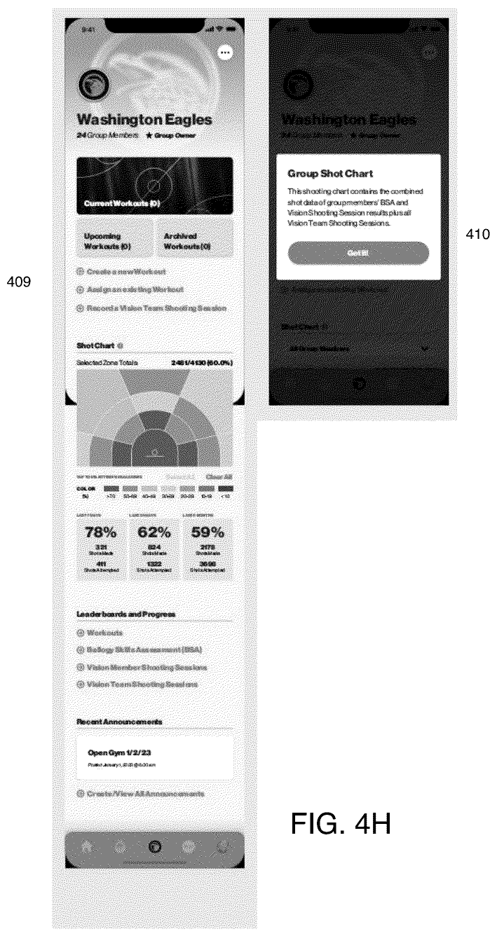

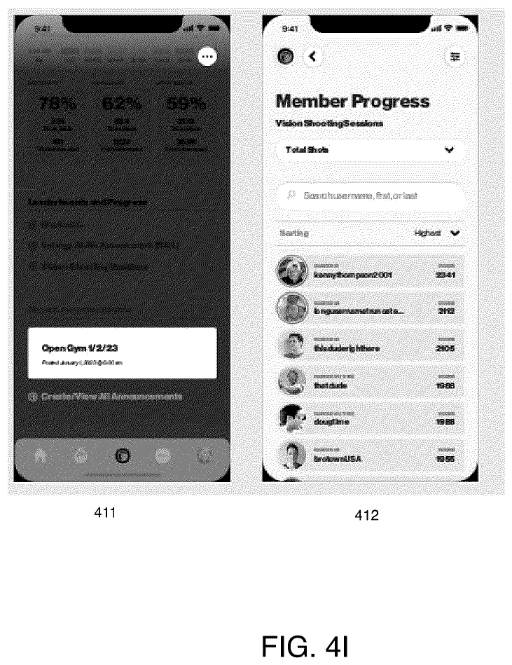

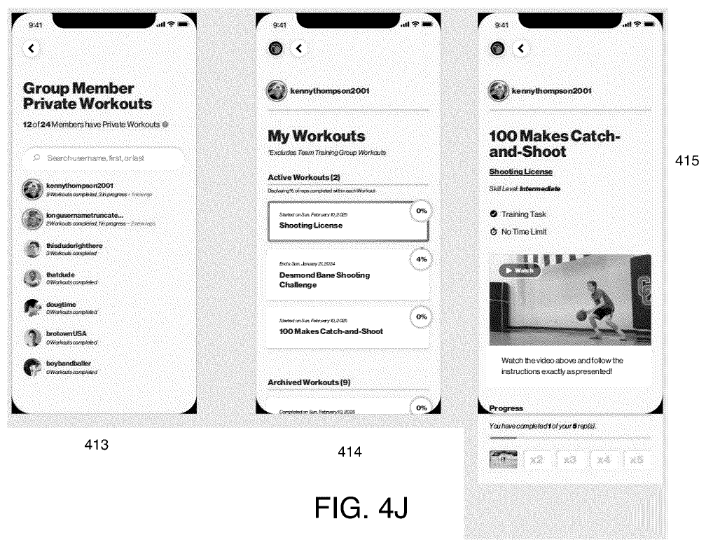

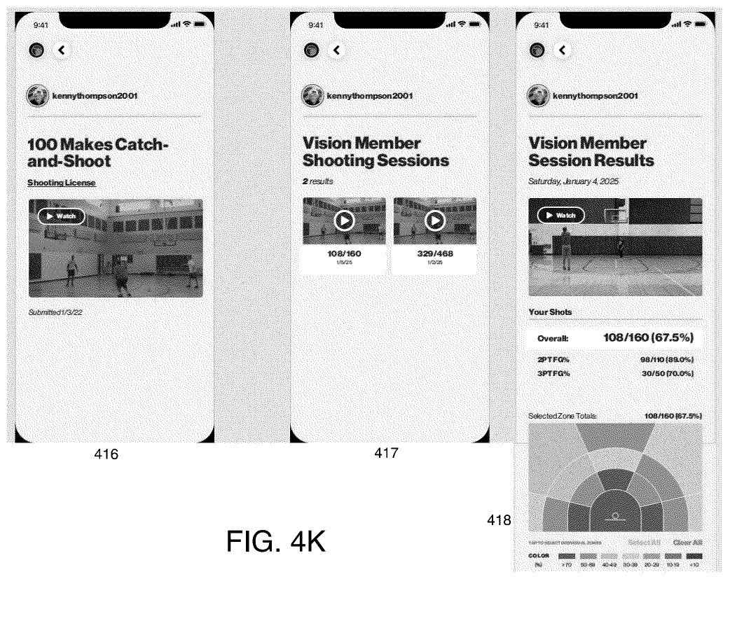

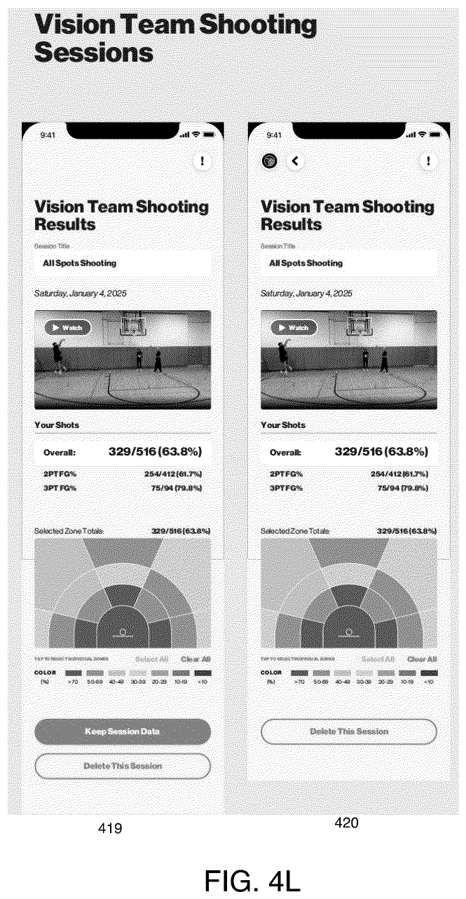

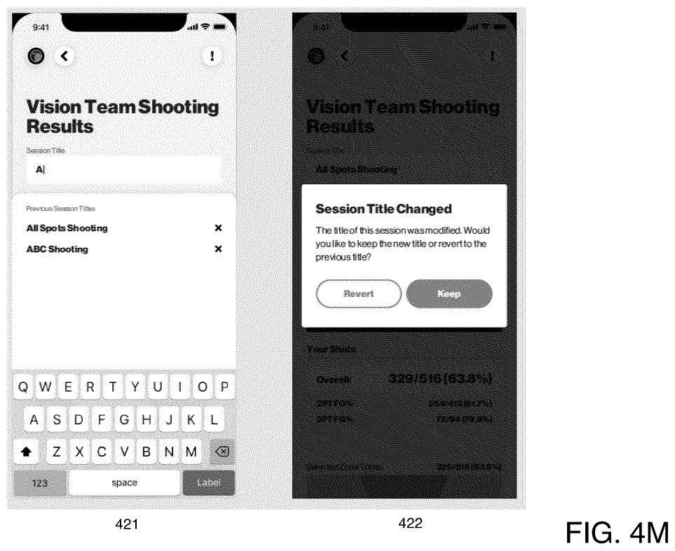

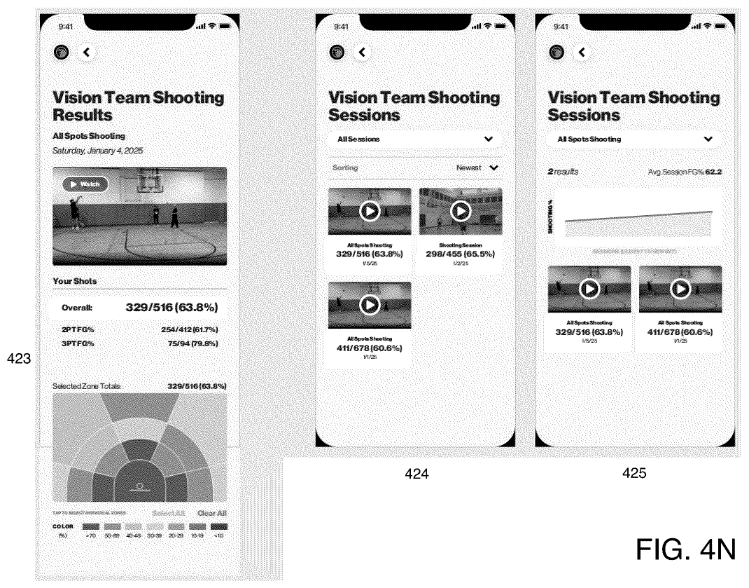

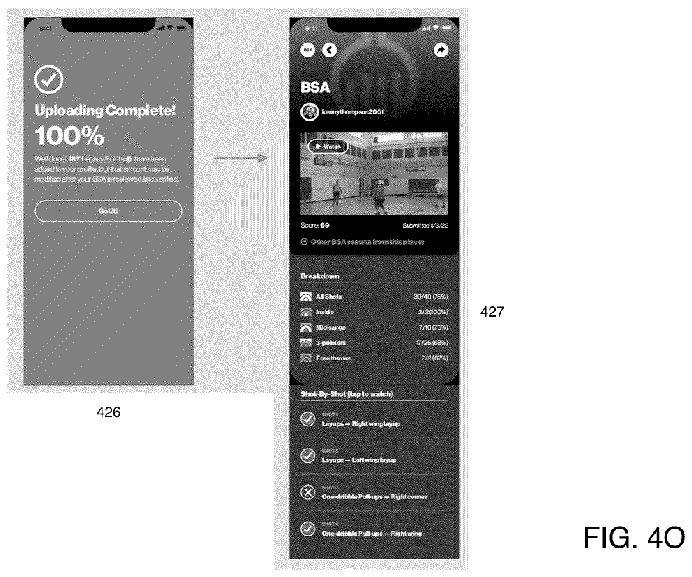

FIG. 3 and associated FIGS. 4A-4O depict example screens (e.g., screenshots) of a graphical user interface including example aspects of a sports social media application (app) pertaining at least in part to computer vision operations, in accordance with one or more embodiments. FIG. 3 and associated FIGS. 4A-4O may include self-explanatory aspects and/or may depict aspects discussed herein in connection with example embodiments. In one or more embodiments, example screens depicted in FIGS. 4A-4O may be displayed on a cellular telephone, for example. Also, for example, screens depicted in FIGS. 4A-4O may depict example features of a graphical user interface (e.g., links, badges, buttons, icons, etc.) by which an app may obtain inputs from a user (e.g., player, coach, official, parent, administrator, etc.), in one or more embodiments.

FIG. 3, along with FIGS. 4A-4O, provide an overview of several screenshots pertaining at least in part to computer vision aspects of an application. For example, as depicted in screen 401 in FIGS. 4A-4B, a user may select a “vision shooting session” button. Responsive at least in part to a selection of a vision shooting session button, screen 402 may be displayed, for example. It may be noted that screen 401 may comprise one or more characteristics discussed above, for example. Also, example screens 401-427 depicted in FIGS. 4A-4O may comprise at least some elements similar to those discussed above in connection with an example “My Workouts” portion of a sports social media application, in one or more embodiments. For example, example screens 401-427 may demonstrate example features that may be incorporated, at least in part, in a “My Workouts” portion of a sports social media application, in one or more embodiments.

In one or more embodiments, at screen 402, instructions may be provided to users on how to set a camera, such as a cellular telephone or other device, for video capture and/or computer vision operations. For example, instructions may include setting up a camera on a tripod or something stable, in one or more embodiments. In one or more embodiments, as depicted at screen 402, a time limit may be specified. For example, a player may wish to test how many shots the player can make within a particular period of time. In other embodiments, a user may manually stop a session rather than rely on a specified amount of time, for example.

In one or more embodiments, to start a vision shooting session, a user (e.g., player, coach, official, etc.) may select a “start your session” button, such as depicted at screen 402, for example.

In one or more embodiments, a session may begin with a calibration operation, such as depicted at screen 403 of FIGS. 4C-4D, for example. Responsive at least in part to a selection of a “start your session” button, for example, a computer vision aspect of an app may align with a basketball court as viewed via a camera. See, for example, screen 403. In one or more embodiments, one or more calibration operations of a computer vision feature may measure a three-point line physically existing on a floor of a basketball court, for example, and/or may further measure a basketball hoop. Successful measurement and/or calibration of such features of a basketball court may result in one or more indications (e.g., green check marks) of successful calibration and/or successful setup being displayed to a user, such as depicted in screen 403, for example. In one or more embodiments, once an indication of successful setup and/or calibration is provided, a user may select a “start recording” button, such as depicted at screen 403 (see FIG. 4D).

In one or more embodiments, a computer vision feature may track shots during a shooting session. See, for example, a shot chart depicted towards the bottom of screens 403 and/or 404. A computer vision feature may, in normal operation, track every made and/or missed shot, and a shot chart may highlight where a player shoots (e.g., location on a basketball court), in one or more embodiments. In one or more embodiments, a made shot may be indicated with a green highlight on a shot chart and/or a missed shot may be indicated with a red highlight on the shot chart, for example.

Also, as depicted at screen 404 of FIG. 4E, a user, such as a player, may review a shooting session once a recording is stopped (e.g., see “stop recording” button on screen 403) and/or may observe a number of makes and/or misses. Additionally, a percentage of made shots may be calculated by a computer vision feature. In one or more embodiments, an overall shooting percentage may be calculated and/or displayed. Further, for example, shooting percentages for individual shooting zones may also be calculated and/or displayed, as depicted at screen 404. In one or more embodiments, screen 404, for example, may also show a heat map, thereby providing to a user a quick assessment of a just-completed shooting session, in one or more embodiments. In one or more embodiments, individual zones may be selected by a user via interactive elements (e.g., heat map) of example screen 404, for example.

In one or more embodiments, opportunities to keep session data or to delete the session data may be provided to a user via respective interactive elements (e.g., buttons) of screen 404, for example. In one or more embodiments, kept session data may be saved to a user's profile for later review and/or for archiving, for example.

Additionally, in one or more embodiments, mechanisms may be provided for a user to indicate inaccurate and/or faulty data. For example, a button may be provided at the top right corner of screen 404 whereby a user, via selection of the button, may alert the application. Responsive to selection of this particular button (denoted by “!”), the application may display screen 405 which may provide the user with interactive elements by which the user may alert the application to faulty and/or inaccurate statistics, for example. Selection of an “alert” button, such as depicted at screen 405, may signal to administrators to investigate, in one or more embodiments. Administrators may analyze and/or investigate the video in question and/or may attempt to make corrections. Acknowledgment of the alert may be provided to the user, such as shown at screen 406 of FIG. 4F, for example. Also, selection by a user of a “delete this session” button of screen 404 may prompt the application to display screen 407 of FIG. 4F, for example, and the user may be provided an opportunity to return to a previous screen or to confirm a desire to delete the session, as indicated.

In one or more embodiments, screen 401 may display a “view all vision shooting sessions” interactive element (e.g., button, link, icon, etc.). Selection of this element may bring a user to screen 408 depicted at FIG. 4G, in one or more embodiments. At screen 408, a user may browse and/or view previously stored vision shooting sessions. In one or more embodiments, at screen 1608 a user may have the ability to look through all stored sessions pertaining to the particular user. Also, screen 408 may provide an opportunity for administrators of the application to display advertisements and/or other content that may be monetarily profitable.

It may be noted that the example shooting session embodiment described above is a fairly straightforward process that may not require a great deal of instruction or guidance for the user. In embodiments, a calibration operation may be relatively autonomous and easy to use. Similarly, the shootaround session itself is essentially a free shoot without much structure with a possible exception of a specified period of time. Further, it may be straightforward for a user to save or delete session data and/or to browse previously stored session video content and/or data, in one or more embodiments.

FIG. 4H depicts example screens 409 and 410 that may depict aspects of a sports social media application pertaining to groups, teams, etc. Screen 409 may depict a group's outline, for example. For groups, any time that a player performs an individual shooting session, a symbol may become highlighted. For example screen 409, a group symbol in the upper left corner may be highlighted with an orange border to signal that a new individual shooting session has occurred. Also, via screen 409, a group owner (e.g., coach, parent, etc.) may be directed to a player's stored private shooting sessions.

Example screen 409 (e.g., home screen for the) may provide an option via a button or link, for example, to record a vision team shooting session as well as anything that individuals of a group may perform on their own, in one or more embodiments. In one or more embodiments, group/team sessions plus Ballogy Skills Assessment (BSA) video content and/or scores may be added within a group shot chart as depicted at screen 409. A pop-up screen 410 may alert a user that the shot chart includes group data, for example. In this manner, a group owner/coach may be able to collectively see the progress of the group owner/coach's team. Armed with such information, adjustments to practice routines, workouts, and/or plays to be utilized during games may be adjusted, for example.

For the example shot chart depicted at screen 409, a representation of a basketball court may be divided into a number of shooting zones. A user may click on a zone to see shooting statistics for that particular zone. In one or more embodiments, a number of zones may be selected and/or deselected in accordance with the user's desires. A deselected zone may be greyed-out, for example. In one or more embodiments, such a shot chart may comprise a collective data snapshot for a coach, for example, to evaluate their team.

In one or more embodiments, as indicated at screen 411 of FIG. 4I, announcements may be created (e.g., by the group owner, coach, etc.), and such announcements (e.g., scheduling, assignments, etc.) may be pushed to the group/team by way of sports social media applications resident on the individual player's electronic devices (e.g., cellular phones), for example.

Also depicted at FIG. 4I is a Member Progress screen 412, which may comprise one or more leaderboards, for example. In one or more embodiments, leaderboards for workouts and/or skills assessments may be provided via screen 412. Additionally, in one or more embodiments, leaderboards for vision shooting sessions may be included for individuals and/or teams.

In one or more embodiments, leaderboards may be utilized by a coach, for example, to see which player is shooting the most, making the highest percentage of shots, etc. A coach may utilize provided metrics to understand what various players are doing on their individual time, for example.

As depicted at FIG. 4J, private workout leaderboards may be provided for individual members of a group/team, as seen in screen 413. Further, screen 414 shows a My Workouts screen, similar in at least some respects to what was explained previously. If a player completes his/her own workout, a coach may have a look to see what their individual players are doing on their own. For example, this may be something that the coach did not assign to them. A coach may click in and see what the particular individual is doing with respect to workouts and such.

Via screen 414, for example, a group owner/coach may select different workouts may be directed to appropriate screens pertaining to the selected workouts. See, for example, screens 415, 416, and/or 417 as depicted at FIGS. 4J-4K. In one or more embodiments, a coach or other group owner may, via the provided screens, determine that a particular player completed a vision shooting session, for example, and may see that, individually, the particular player shot a particular score. For example, as shown at example screen 418, a coach may observe that a particular player shot 108 of 160, for example, and with that information may interact with the player to provide praise, instruction, etc. As also depicted at screen 418, coaches may view vision session video content for particular players, for example, in addition to being provided with shooting statistics. Such example features may help coaches to encourage and/or instruct players and/or to help keep players accountable. For example, a coach may be able to determine whether players are intrinsically motivated or not, and the coach may provide appropriate instruction.

FIG. 4L depicts example screens 419-420, showing vision team shooting session data and/or video content. For team sessions, the process may proceed in a manner similar in at least some respects to that explained above for individual vision shooting sessions in terms of setup, calibration, recording, saving, deleting, etc. Set up and/or calibration may be performed similar to what is shown in FIGS. 4C-4D, for example. For team sessions, video content from the session may be analyzed via AI/machine-learning to determine collective shooting statistics for the team.

Team vision shooting session results may be displayed on screen 419/420, for example. A shot chart and/or provided statistics may provide readily digestible information pertaining to a team's shooting performance during a team shooting session, and a coach may track and/or measure performance in real-time or near real-time, in one or more embodiments.

Once a vision team shooting session is completed and stored/saved, it may be accessed via a vision team shooting results screen, such as shown at screen 421, as depicted at FIG. M, for example. A search feature may be provided to allow a coach access to previous vision team shooting results. Also, as shown at screen 422, a group owner/coach may add or change a session title if desired, for example.

FIG. 4N depicts example screens 423, 424, and 425 showing vision team shooting results. In some circumstances, a coach may want to see all of the practice shooting drills for the past year, for example. In one or more embodiments, the coach may review the specified sessions (e.g., may be specified via a drop-down menu) with their various titles displayed, such as shown at screen 424, for example. A computer vision feature of an application may calculate differences between various shooting sessions, for example, and/or may detect and/or predict performance trends, in one or more embodiments.

For example, a coach may wish to know how a team performed in “all spots” shooting sessions over a period of time and/or as measured against other sessions. A coach may readily discern deltas between different sessions, in one or more embodiments. As shown in screen 425, for example, a trend line may be calculated and/or displayed, for example.

In one or more embodiments, computer vision feature implementation may make BSA scores available much quicker-essentially in real-time or near real-time, for example. As depicted at FIG. 4O, an example screen 426 may indicate that a BSA upload has completed. Screen 425 may further indicate, for example, that the user has been awarded points (e.g., legacy points). For example, after a player has finished a Ballogy skills assessment (BSA), the player may be awarded 100 legacy points, in addition to whatever the player's BSA score was, in one or more embodiments. Of course, subject matter is not limited in scope in these respects.

In one or more embodiments, BSA results may be displayed on screen 427, for example. In one or more embodiments, computer vision operations may count makes and misses for various shooting zones and/or may calculate scores. As previously discussed, for BSA, layups may be counted as one, mid-range may be counted as two, three-pointers may be counted as three, and/or the free throws may be counted as one. Such scores may be analyzed and/or calculated autonomously by computer vision features, including AI/machine-learning operations, within a sports social media application.

In embodiments, an option may be included whereby a group owner/coach may restrict a player's permission to delete workout results and/or BSA results. This may help a coach, for example, understand the true progress for a player that might not be appreciated if a player is able to pick and choose which results to keep and which to delete.

In embodiments, players may be incentivized to keep workout and/or BSA results via a rewards system. For example, for a 10,000 shot club, a coach may ask players to get up 10,000 shots over the course of a period of time (e.g., over the course of a summer/offseason). To have a player deleting workouts and/or results would tend to retard a player's progress towards 10,000 shots. To help address this circumstance, for implementations wherein a player may be restricted in how many or how often workout results and/or BSA results may be deleted, the player may be incentivized to prepare for the workout/BSA properly so as to minimize the chances of a poor performance, in one or more embodiments.

In other embodiments, deletion of workouts and/or BSA may be allowed and/or a record may be kept of any deletions and/or a record may be kept of a number of starts and/or stops. Some embodiments may track merely track the fact that they started and stopped. For example, a particular player has twenty starts and stops, but they only recorded two sessions. Such information may be helpful to coaches, parents, administrators, etc.

In some embodiments, workouts may be deleted but BSA results may not be deleted because the BSA is more of an evaluation. In some embodiments, a specified number of scores may be utilized to assess a player. For example, even though a player may not be permitted to delete BSA results, only the top two scores, for example, would be submitted to colleges or other interested parties, for example. See discussion above regarding how BSA scores may be generated, for one or more embodiments.

In the context of the present patent application, the term “connection,” the term “component” and/or similar terms are intended to be physical, but are not necessarily always tangible. Whether or not these terms refer to tangible subject matter, thus, may vary in a particular context of usage. As an example, a tangible connection and/or tangible connection path may be made, such as by a tangible, electrical connection, such as an electrically conductive path comprising metal or other conductor, that is able to conduct electrical current between two tangible components. Likewise, a tangible connection path may be at least partially affected and/or controlled, such that, as is typical, a tangible connection path may be open or closed, at times resulting from influence of one or more externally derived signals, such as external currents and/or voltages, such as for an electrical switch. Non-limiting illustrations of an electrical switch include a transistor, a diode, etc. However, a “connection” and/or “component,” in a particular context of usage, likewise, although physical, can also be non-tangible, such as a connection between a client and a server over a network, particularly a wireless network, which generally refers to the ability for the client and server to transmit, receive, and/or exchange communications, as discussed in more detail later.

In a particular context of usage, such as a particular context in which tangible components are being discussed, therefore, the terms “coupled” and “connected” are used in a manner so that the terms are not synonymous. Similar terms may also be used in a manner in which a similar intention is exhibited. Thus, “connected” is used to indicate that two or more tangible components and/or the like, for example, are tangibly in direct physical contact. Thus, using the previous example, two tangible components that are electrically connected are physically connected via a tangible electrical connection, as previously discussed. However, “coupled,” is used to mean that potentially two or more tangible components are tangibly in direct physical contact. Nonetheless, “coupled” is also used to mean that two or more tangible components and/or the like are not necessarily tangibly in direct physical contact, but are able to co-operate, liaise, and/or interact, such as, for example, by being “optically coupled.” Likewise, the term “coupled” is also understood to mean indirectly connected. It is further noted, in the context of the present patent application, since memory, such as a memory component and/or memory states, is intended to be non-transitory, the term physical, at least if used in relation to memory necessarily implies that such memory components and/or memory states, continuing with the example, are tangible.

Additionally, in the present patent application, in a particular context of usage, such as a situation in which tangible components (and/or similarly, tangible materials) are being discussed, a distinction exists between being “on” and being “over.” As an example, deposition of a substance “on” a substrate refers to a deposition involving direct physical and tangible contact without an intermediary, such as an intermediary substance, between the substance deposited and the substrate in this latter example; nonetheless, deposition “over” a substrate, while understood to potentially include deposition “on” a substrate (since being “on” may also accurately be described as being “over”), is understood to include a situation in which one or more intermediaries, such as one or more intermediary substances, are present between the substance deposited and the substrate so that the substance deposited is not necessarily in direct physical and tangible contact with the substrate.

A similar distinction is made in an appropriate particular context of usage, such as in which tangible materials and/or tangible components are discussed, between being “beneath” and being “under.” While “beneath,” in such a particular context of usage, is intended to necessarily imply physical and tangible contact (similar to “on,” as just described), “under” potentially includes a situation in which there is direct physical and tangible contact, but does not necessarily imply direct physical and tangible contact, such as if one or more intermediaries, such as one or more intermediary substances, are present. Thus, “on” is understood to mean “immediately over” and “beneath” is understood to mean “immediately under.”

It is likewise appreciated that terms such as “over” and “under” are understood in a similar manner as the terms “up,” “down,” “top,” “bottom,” and so on, previously mentioned. These terms may be used to facilitate discussion, but are not intended to necessarily restrict scope of claimed subject matter. For example, the term “over,” as an example, is not meant to suggest that claim scope is limited to only situations in which an embodiment is right side up, such as in comparison with the embodiment being upside down, for example. An example includes a flip chip, as one illustration, in which, for example, orientation at various times (e.g., during fabrication) may not necessarily correspond to orientation of a final product. Thus, if an object, as an example, is within applicable claim scope in a particular orientation, such as upside down, as one example, likewise, it is intended that the latter also be interpreted to be included within applicable claim scope in another orientation, such as right side up, again, as an example, and vice-versa, even if applicable literal claim language has the potential to be interpreted otherwise. Of course, again, as always has been the case in the specification of a patent application, particular context of description and/or usage provides helpful guidance regarding reasonable inferences to be drawn.

Unless otherwise indicated, in the context of the present patent application, the term “or” if used to associate a list, such as A, B, or C, is intended to mean A, B, and C, here used in the inclusive sense, as well as A, B, or C, here used in the exclusive sense. With this understanding, “and” is used in the inclusive sense and intended to mean A, B, and C; whereas “and/or” can be used in an abundance of caution to make clear that all of the foregoing meanings are intended, although such usage is not required. In addition, the term “one or more” and/or similar terms is used to describe any feature, structure, characteristic, and/or the like in the singular, “and/or” is also used to describe a plurality and/or some other combination of features, structures, characteristics, and/or the like. Likewise, the term “based on” and/or similar terms are understood as not necessarily intending to convey an exhaustive list of factors, but to allow for existence of additional factors not necessarily expressly described.

Furthermore, it is intended, for a situation that relates to implementation of claimed subject matter and is subject to testing, measurement, and/or specification regarding degree, that the particular situation be understood in the following manner. As an example, in a given situation, assume a value of a physical property is to be measured. If alternatively reasonable approaches to testing, measurement, and/or specification regarding degree, at least with respect to the property, continuing with the example, is reasonably likely to occur to one of ordinary skill, at least for implementation purposes, claimed subject matter is intended to cover those alternatively reasonable approaches unless otherwise expressly indicated. As an example, if a plot of measurements over a region is produced and implementation of claimed subject matter refers to employing a measurement of slope over the region, but a variety of reasonable and alternative techniques to estimate the slope over that region exist, claimed subject matter is intended to cover those reasonable alternative techniques unless otherwise expressly indicated.

To the extent claimed subject matter is related to one or more particular measurements, such as with regard to physical manifestations capable of being measured physically, such as, without limit, temperature, pressure, voltage, current, electromagnetic radiation, etc., it is believed that claimed subject matter does not fall within the abstract idea judicial exception to statutory subject matter. Rather, it is asserted, that physical measurements are not mental steps and, likewise, are not abstract ideas.

It is noted, nonetheless, that a typical measurement model employed is that one or more measurements may respectively comprise a sum of at least two components. Thus, for a given measurement, for example, one component may comprise a deterministic component, which in an ideal sense, may comprise a physical value (e.g., sought via one or more measurements), often in the form of one or more signals, signal samples and/or states, and one component may comprise a random component, which may have a variety of sources that may be challenging to quantify. At times, for example, lack of measurement precision may affect a given measurement. Thus, for claimed subject matter, a statistical or stochastic model may be used in addition to a deterministic model as an approach to identification and/or prediction regarding one or more measurement values that may relate to claimed subject matter.

For example, a relatively large number of measurements may be collected to better estimate a deterministic component. Likewise, if measurements vary, which may typically occur, it may be that some portion of a variance may be explained as a deterministic component, while some portion of a variance may be explained as a random component. Typically, it is desirable to have stochastic variance associated with measurements be relatively small, if feasible. That is, typically, it may be preferable to be able to account for a reasonable portion of measurement variation in a deterministic manner, rather than a stochastic matter as an aid to identification and/or predictability.

Along these lines, a variety of techniques have come into use so that one or more measurements may be processed to better estimate an underlying deterministic component, as well as to estimate potentially random components. These techniques, of course, may vary with details surrounding a given situation. Typically, however, more complex problems may involve use of more complex techniques. In this regard, as alluded to above, one or more measurements of physical manifestations may be modeled deterministically and/or stochastically. Employing a model permits collected measurements to potentially be identified and/or processed, and/or potentially permits estimation and/or prediction of an underlying deterministic component, for example, with respect to later measurements to be taken. A given estimate may not be a perfect estimate; however, in general, it is expected that on average one or more estimates may better reflect an underlying deterministic component, for example, if random components that may be included in one or more obtained measurements, are considered. Practically speaking, of course, it is desirable to be able to generate, such as through estimation approaches, a physically meaningful model of processes affecting measurements to be taken.

In some situations, however, as indicated, potential influences may be complex. Therefore, seeking to understand appropriate factors to consider may be particularly challenging. In such situations, it is, therefore, not unusual to employ heuristics with respect to generating one or more estimates. Heuristics refers to use of experience related approaches that may reflect realized processes and/or realized results, such as with respect to use of historical measurements, for example. Heuristics, for example, may be employed in situations where more analytical approaches may be overly complex and/or nearly intractable. Thus, regarding claimed subject matter, an innovative feature may include, in an example embodiment, heuristics that may be employed, for example, to estimate and/or predict one or more measurements.

It is further noted that the terms “type” and/or “like,” if used, such as with a feature, structure, characteristic, and/or the like, using “optical” or “electrical” as simple examples, means at least partially of and/or relating to the feature, structure, characteristic, and/or the like in such a way that presence of minor variations, even variations that might otherwise not be considered fully consistent with the feature, structure, characteristic, and/or the like, do not in general prevent the feature, structure, characteristic, and/or the like from being of a “type” and/or being “like,” (such as being an “optical-type” or being “optical-like,” for example) if the minor variations are sufficiently minor so that the feature, structure, characteristic, and/or the like would still be considered to be substantially present with such variations also present. Thus, continuing with this example, the terms optical-type and/or optical-like properties are necessarily intended to include optical properties. Likewise, the terms electrical-type and/or electrical-like properties, as another example, are necessarily intended to include electrical properties. It should be noted that the specification of the present patent application merely provides one or more illustrative examples and claimed subject matter is intended to not be limited to one or more illustrative examples; however, again, as has always been the case with respect to the specification of a patent application, particular context of description and/or usage provides helpful guidance regarding reasonable inferences to be drawn.

With advances in technology, it has become more typical to employ distributed computing and/or communication approaches in which portions of a process, such as signal processing of signal samples, for example, may be allocated among various devices, including one or more client devices and/or one or more server devices, via a computing and/or communications network, for example. A network may comprise two or more devices, such as network devices and/or computing devices, and/or may couple devices, such as network devices and/or computing devices, so that signal communications, such as in the form of signal packets and/or signal frames (e.g., comprising one or more signal samples), for example, may be exchanged, such as between a server device and/or a client device, as well as other types of devices, including between wired and/or wireless devices coupled via a wired and/or wireless network, for example.

An example of a distributed computing system comprises the so-called Hadoop distributed computing system, which employs a map-reduce type of architecture. In the context of the present patent application, the terms map-reduce architecture and/or similar terms are intended to refer to a distributed computing system implementation and/or embodiment for processing and/or for generating larger sets of signal samples employing map and/or reduce operations for a parallel, distributed process performed over a network of devices. A map operation and/or similar terms refer to processing of signals (e.g., signal samples) to generate one or more key-value pairs and to distribute the one or more pairs to one or more devices of the system (e.g., network). A reduce operation and/or similar terms refer to processing of signals (e.g., signal samples) via a summary operation (e.g., such as counting the number of students in a queue, yielding name frequencies, etc.). A system may employ such an architecture, such as by marshaling distributed server devices, executing various tasks in parallel, and/or managing communications, such as signal transfers, between various parts of the system (e.g., network), in an embodiment. As mentioned, one non-limiting, but well-known, example comprises the Hadoop distributed computing system. It refers to an open source implementation and/or embodiment of a map-reduce type architecture (available from the Apache Software Foundation, 1901 Munsey Drive, Forrest Hill, MD, 21050-2747), but may include other aspects, such as the Hadoop distributed file system (HDFS) (available from the Apache Software Foundation, 1901 Munsey Drive, Forrest Hill, MD, 21050-2747). In general, therefore, “Hadoop” and/or similar terms (e.g., “Hadoop-type,” etc.) refer to an implementation and/or embodiment of a scheduler for executing larger processing jobs using a map-reduce architecture over a distributed system. Furthermore, in the context of the present patent application, use of the term “Hadoop” is intended to include versions, presently known and/or to be later developed.

In the context of the present patent application, the term network device refers to any device capable of communicating via and/or as part of a network and may comprise a computing device. While network devices may be capable of communicating signals (e.g., signal packets and/or frames), such as via a wired and/or wireless network, they may also be capable of performing operations associated with a computing device, such as arithmetic and/or logic operations, processing and/or storing operations (e.g., storing signal samples), such as in memory as tangible, physical memory states, and/or may, for example, operate as a server device and/or a client device in various embodiments. Network devices capable of operating as a server device, a client device and/or otherwise, may include, as examples, dedicated rack-mounted servers, desktop computers, laptop computers, set top boxes, tablets, netbooks, smart phones, wearable devices, integrated devices combining two or more features of the foregoing devices, and/or the like, or any combination thereof. As mentioned, signal packets and/or frames, for example, may be exchanged, such as between a server device and/or a client device, as well as other types of devices, including between wired and/or wireless devices coupled via a wired and/or wireless network, for example, or any combination thereof. It is noted that the terms, server, server device, server computing device, server computing platform and/or similar terms are used interchangeably. Similarly, the terms client, client device, client computing device, client computing platform and/or similar terms are also used interchangeably. While in some instances, for ease of description, these terms may be used in the singular, such as by referring to a “client device” or a “server device,” the description is intended to encompass one or more client devices and/or one or more server devices, as appropriate. Along similar lines, references to a “database” are understood to mean, one or more databases and/or portions thereof, as appropriate.

It should be understood that for ease of description, a network device (also referred to as a networking device) may be embodied and/or described in terms of a computing device and vice-versa. However, it should further be understood that this description should in no way be construed so that claimed subject matter is limited to one embodiment, such as only a computing device and/or only a network device, but, instead, may be embodied as a variety of devices or combinations thereof, including, for example, one or more illustrative examples.

A network may also include now known, and/or to be later developed arrangements, derivatives, and/or improvements, including, for example, past, present and/or future mass storage, such as network attached storage (NAS), a storage area network (SAN), and/or other forms of device readable media, for example. A network may include a portion of the Internet, one or more local area networks (LANs), one or more wide area networks (WANs), wire-line type connections, wireless type connections, other connections, or any combination thereof. Thus, a network may be worldwide in scope and/or extent. Likewise, sub-networks, such as may employ differing architectures and/or may be substantially compliant and/or substantially compatible with differing protocols, such as network computing and/or communications protocols (e.g., network protocols), may interoperate within a larger network.

In the context of the present patent application, the term sub-network and/or similar terms, if used, for example, with respect to a network, refers to the network and/or a part thereof. Sub-networks may also comprise links, such as physical links, connecting and/or coupling nodes, so as to be capable to communicate signal packets and/or frames between devices of particular nodes, including via wired links, wireless links, or combinations thereof. Various types of devices, such as network devices and/or computing devices, may be made available so that device interoperability is enabled and/or, in at least some instances, may be transparent. In the context of the present patent application, the term “transparent,” if used with respect to devices of a network, refers to devices communicating via the network in which the devices are able to communicate via one or more intermediate devices, such as one or more intermediate nodes, but without the communicating devices necessarily specifying the one or more intermediate nodes and/or the one or more intermediate devices of the one or more intermediate nodes and/or, thus, may include within the network the devices communicating via the one or more intermediate nodes and/or the one or more intermediate devices of the one or more intermediate nodes, but may engage in signal communications as if such intermediate nodes and/or intermediate devices are not necessarily involved. For example, a router may provide a link and/or connection between otherwise separate and/or independent LANs.

In the context of the present patent application, a “private network” refers to a particular, limited set of devices, such as network devices and/or computing devices, able to communicate with other devices, such as network devices and/or computing devices, in the particular, limited set, such as via signal packet and/or signal frame communications, for example, without a need for re-routing and/or redirecting signal communications. A private network may comprise a stand-alone network; however, a private network may also comprise a subset of a larger network, such as, for example, without limitation, all or a portion of the Internet. Thus, for example, a private network “in the cloud” may refer to a private network that comprises a subset of the Internet. Although signal packet and/or frame communications (e.g. signal communications) may employ intermediate devices of intermediate nodes to exchange signal packets and/or signal frames, those intermediate devices may not necessarily be included in the private network by not being a source or designated destination for one or more signal packets and/or signal frames, for example. It is understood in the context of the present patent application that a private network may direct outgoing signal communications to devices not in the private network, but devices outside the private network may not necessarily be able to direct inbound signal communications to devices included in the private network.

The Internet refers to a decentralized global network of interoperable networks that comply with the Internet Protocol (IP). It is noted that there are several versions of the Internet Protocol. The term Internet Protocol, IP, and/or similar terms are intended to refer to any version, now known and/or to be later developed. The Internet includes local area networks (LANs), wide area networks (WANs), wireless networks, and/or long haul public networks that, for example, may allow signal packets and/or frames to be communicated between LANs. The term World Wide Web (WWW or Web) and/or similar terms may also be used, although it refers to a part of the Internet that complies with the Hypertext Transfer Protocol (HTTP). For example, network devices may engage in an HTTP session through an exchange of appropriately substantially compatible and/or substantially compliant signal packets and/or frames. It is noted that there are several versions of the Hypertext Transfer Protocol. The term Hypertext Transfer Protocol, HTTP, and/or similar terms are intended to refer to any version, now known and/or to be later developed. It is likewise noted that in various places in this document substitution of the term Internet with the term World Wide Web (“Web”) may be made without a significant departure in meaning and may, therefore, also be understood in that manner if the statement would remain correct with such a substitution.

Although claimed subject matter is not in particular limited in scope to the Internet and/or to the Web; nonetheless, the Internet and/or the Web may without limitation provide a useful example of an embodiment at least for purposes of illustration. As indicated, the Internet and/or the Web may comprise a worldwide system of interoperable networks, including interoperable devices within those networks. The Internet and/or Web has evolved to a public, self-sustaining facility accessible to potentially billions of people or more worldwide. Also, in an embodiment, and as mentioned above, the terms “WWW” and/or “Web” refer to a part of the Internet that complies with the Hypertext Transfer Protocol. The Internet and/or the Web, therefore, in the context of the present patent application, may comprise a service that organizes stored digital content, such as, for example, text, images, video, etc., through the use of hypermedia, for example. It is noted that a network, such as the Internet and/or Web, may be employed to store electronic files and/or electronic documents.

The term electronic file and/or the term electronic document are used throughout this document to refer to a set of stored memory states and/or a set of physical signals associated in a manner so as to thereby at least logically form a file (e.g., electronic) and/or an electronic document. That is, it is not meant to implicitly reference a particular syntax, format and/or approach used, for example, with respect to a set of associated memory states and/or a set of associated physical signals. If a particular type of file storage format and/or syntax, for example, is intended, it is referenced expressly. It is further noted an association of memory states, for example, may be in a logical sense and not necessarily in a tangible, physical sense. Thus, although signal and/or state components of a file and/or an electronic document, for example, are to be associated logically, storage thereof, for example, may reside in one or more different places in a tangible, physical memory, in an embodiment.

A Hyper Text Markup Language (“HTML”), for example, may be utilized to specify digital content and/or to specify a format thereof, such as in the form of an electronic file and/or an electronic document, such as a Web page, Web site, etc., for example. An Extensible Markup Language (“XML”) may also be utilized to specify digital content and/or to specify a format thereof, such as in the form of an electronic file and/or an electronic document, such as a Web page, Web site, etc., in an embodiment. Of course, HTML and/or XML are merely examples of “markup” languages, provided as non-limiting illustrations. Furthermore, HTML and/or XML are intended to refer to any version, now known and/or to be later developed, of these languages. Likewise, claimed subject matter are not intended to be limited to examples provided as illustrations, of course.

In the context of the present patent application, the term “Web site” and/or similar terms refer to Web pages that are associated electronically to form a particular collection thereof. Also, in the context of the present patent application, “Web page” and/or similar terms refer to an electronic file and/or an electronic document accessible via a network, including by specifying a uniform resource locator (URL) for accessibility via the Web, in an example embodiment. As alluded to above, in one or more embodiments, a Web page may comprise digital content coded (e.g., via computer instructions) using one or more languages, such as, for example, markup languages, including HTML and/or XML, although claimed subject matter is not limited in scope in this respect. Also, in one or more embodiments, application developers may write code (e.g., computer instructions) in the form of JavaScript (or other programming languages), for example, executable by a computing device to provide digital content to populate an electronic document and/or an electronic file in an appropriate format, such as for use in a particular application, for example. Use of the term “JavaScript” and/or similar terms intended to refer to one or more particular programming languages are intended to refer to any version of the one or more programming languages identified, now known and/or to be later developed. Thus, JavaScript is merely an example programming language. As was mentioned, claimed subject matter is not intended to be limited to examples and/or illustrations.

In the context of the present patent application, the terms “entry,” “electronic entry,” “document,” “electronic document,” “content,”, “digital content,” “item,” and/or similar terms are meant to refer to signals and/or states in a physical format, such as a digital signal and/or digital state format, e.g., that may be perceived by a user if displayed, played, tactilely generated, etc. and/or otherwise executed by a device, such as a digital device, including, for example, a computing device, but otherwise might not necessarily be readily perceivable by humans (e.g., if in a digital format). Likewise, in the context of the present patent application, digital content provided to a user in a form so that the user is able to readily perceive the underlying content itself (e.g., content presented in a form consumable by a human, such as hearing audio, feeling tactile sensations and/or seeing images, as examples) is referred to, with respect to the user, as “consuming” digital content, “consumption” of digital content, “consumable” digital content and/or similar terms. For one or more embodiments, an electronic document and/or an electronic file may comprise a Web page of code (e.g., computer instructions) in a markup language executed or to be executed by a computing and/or networking device, for example. In another embodiment, an electronic document and/or electronic file may comprise a portion and/or a region of a Web page. However, claimed subject matter is not intended to be limited in these respects.

Also, for one or more embodiments, an electronic document and/or electronic file may comprise a number of components. As previously indicated, in the context of the present patent application, a component is physical, but is not necessarily tangible. As an example, components with reference to an electronic document and/or electronic file, in one or more embodiments, may comprise text, for example, in the form of physical signals and/or physical states (e.g., capable of being physically displayed). Typically, memory states, for example, comprise tangible components, whereas physical signals are not necessarily tangible, although signals may become (e.g., be made) tangible, such as if appearing on a tangible display, for example, as is not uncommon. Also, for one or more embodiments, components with reference to an electronic document and/or electronic file may comprise a graphical object, such as, for example, an image, such as a digital image, and/or sub-objects, including attributes thereof, which, again, comprise physical signals and/or physical states (e.g., capable of being tangibly displayed). In an embodiment, digital content may comprise, for example, text, images, audio, video, and/or other types of electronic documents and/or electronic files, including portions thereof, for example.

Also, in the context of the present patent application, the term “parameters” (e.g., one or more parameters), “values” (e.g., one or more values), “symbols” (e.g., one or more symbols) “bits” (e.g., one or more bits), “elements” (e.g., one or more elements), “characters” (e.g., one or more characters), “numbers” (e.g., one or more numbers), “numerals” (e.g., one or more numerals) or “measurements” (e.g., one or more measurements) refer to material descriptive of a collection of signals, such as in one or more electronic documents and/or electronic files, and exist in the form of physical signals and/or physical states, such as memory states. For example, one or more parameters, values, symbols, bits, elements, characters, numbers, numerals or measurements, such as referring to one or more aspects of an electronic document and/or an electronic file comprising an image, may include, as examples, time of day at which an image was captured, latitude and longitude of an image capture device, such as a camera, for example, etc. In another example, one or more parameters, values, symbols, bits, elements, characters, numbers, numerals or measurements, relevant to digital content, such as digital content comprising a technical article, as an example, may include one or more authors, for example. Claimed subject matter is intended to embrace meaningful, descriptive parameters, values, symbols, bits, elements, characters, numbers, numerals or measurements in any format, so long as the one or more parameters, values, symbols, bits, elements, characters, numbers, numerals or measurements comprise physical signals and/or states, which may include, as parameter, value, symbol bits, elements, characters, numbers, numerals or measurements examples, collection name (e.g., electronic file and/or electronic document identifier name), technique of creation, purpose of creation, time and date of creation, logical path if stored, coding formats (e.g., type of computer instructions, such as a markup language) and/or standards and/or specifications used so as to be protocol compliant (e.g., meaning substantially compliant and/or substantially compatible) for one or more uses, and so forth.

Signal packet communications and/or signal frame communications, also referred to as signal packet transmissions and/or signal frame transmissions (or merely “signal packets” or “signal frames”), may be communicated between nodes of a network, where a node may comprise one or more network devices and/or one or more computing devices, for example. As an illustrative example, but without limitation, a node may comprise one or more sites employing a local network address, such as in a local network address space. Likewise, a device, such as a network device and/or a computing device, may be associated with that node. It is also noted that in the context of this patent application, the term “transmission” is intended as another term for a type of signal communication that may occur in any one of a variety of situations. Thus, it is not intended to imply a particular directionality of communication and/or a particular initiating end of a communication path for the “transmission” communication. For example, the mere use of the term in and of itself is not intended, in the context of the present patent application, to have particular implications with respect to the one or more signals being communicated, such as, for example, whether the signals are being communicated “to” a particular device, whether the signals are being communicated “from” a particular device, and/or regarding which end of a communication path may be initiating communication, such as, for example, in a “push type” of signal transfer or in a “pull type” of signal transfer. In the context of the present patent application, push and/or pull type signal transfers are distinguished by which end of a communications path initiates signal transfer.

Thus, a signal packet and/or frame may, as an example, be communicated via a communication channel and/or a communication path, such as comprising a portion of the Internet and/or the Web, from a site via an access node coupled to the Internet or vice-versa. Likewise, a signal packet and/or frame may be forwarded via network nodes to a target site coupled to a local network, for example. A signal packet and/or frame communicated via the Internet and/or the Web, for example, may be routed via a path, such as either being “pushed” or “pulled,” comprising one or more gateways, servers, etc. that may, for example, route a signal packet and/or frame, such as, for example, substantially in accordance with a target and/or destination address and availability of a network path of network nodes to the target and/or destination address. Although the Internet and/or the Web comprise a network of interoperable networks, not all of those interoperable networks are necessarily available and/or accessible to the public.

In the context of the particular patent application, a network protocol, such as for communicating between devices of a network, may be characterized, at least in part, substantially in accordance with a layered description, such as the so-called Open Systems Interconnection (OSI) seven layer type of approach and/or description. A network computing and/or communications protocol (also referred to as a network protocol) refers to a set of signaling conventions, such as for communication transmissions, for example, as may take place between and/or among devices in a network. In the context of the present patent application, the term “between” and/or similar terms are understood to include “among” if appropriate for the particular usage and vice-versa. Likewise, in the context of the present patent application, the terms “compatible with,” “comply with” and/or similar terms are understood to respectively include substantial compatibility and/or substantial compliance.

A network protocol, such as protocols characterized substantially in accordance with the aforementioned OSI description, has several layers. These layers are referred to as a network stack. Various types of communications (e.g., transmissions), such as network communications, may occur across various layers. A lowest level layer in a network stack, such as the so-called physical layer, may characterize how symbols (e.g., bits and/or bytes) are communicated as one or more signals (and/or signal samples) via a physical medium (e.g., twisted pair copper wire, coaxial cable, fiber optic cable, wireless air interface, combinations thereof, etc.). Progressing to higher-level layers in a network protocol stack, additional operations and/or features may be available via engaging in communications that are substantially compatible and/or substantially compliant with a particular network protocol at these higher-level layers. For example, higher-level layers of a network protocol may, for example, affect device permissions, user permissions, etc.

A network and/or sub-network, in an embodiment, may communicate via signal packets and/or signal frames, such as via participating digital devices and may be substantially compliant and/or substantially compatible with, but is not limited to, now known and/or to be developed, versions of any of the following network protocol stacks: ARCNET, AppleTalk, ATM, Bluetooth, DECnet, Ethernet, FDDI, Frame Relay, HIPPI, IEEE 1394, IEEE 802.11, IEEE-488, Internet Protocol Suite, IPX, Myrinet, OSI Protocol Suite, QsNet, RS-232, SPX, System Network Architecture, Token Ring, USB, and/or X.25. A network and/or sub-network may employ, for example, a version, now known and/or later to be developed, of the following: TCP/IP, UDP, DECnet, NetBEUI, IPX, AppleTalk and/or the like. Versions of the Internet Protocol (IP) may include IPv4, IPv6, and/or other later to be developed versions.

Regarding aspects related to a network, including a communications and/or computing network, a wireless network may couple devices, including client devices, with the network. A wireless network may employ stand-alone, ad-hoc networks, mesh networks, Wireless LAN (WLAN) networks, cellular networks, and/or the like. A wireless network may further include a system of terminals, gateways, routers, and/or the like coupled by wireless radio links, and/or the like, which may move freely, randomly and/or organize themselves arbitrarily, such that network topology may change, at times even rapidly. A wireless network may further employ a plurality of network access technologies, including a version of Long Term Evolution (LTE), WLAN, Wireless Router (WR) mesh, 2nd, 3rd, or 4th generation (2G, 3G, 4G, or 5G) cellular technology and/or the like, whether currently known and/or to be later developed. Network access technologies may enable wide area coverage for devices, such as computing devices and/or network devices, with varying degrees of mobility, for example.

A network may enable radio frequency and/or other wireless type communications via a wireless network access technology and/or air interface, such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, ultra-wideband (UWB), 802.11b/g/n, and/or the like. A wireless network may include virtually any type of now known and/or to be developed wireless communication mechanism and/or wireless communications protocol by which signals may be communicated between devices, between networks, within a network, and/or the like, including the foregoing, of course.

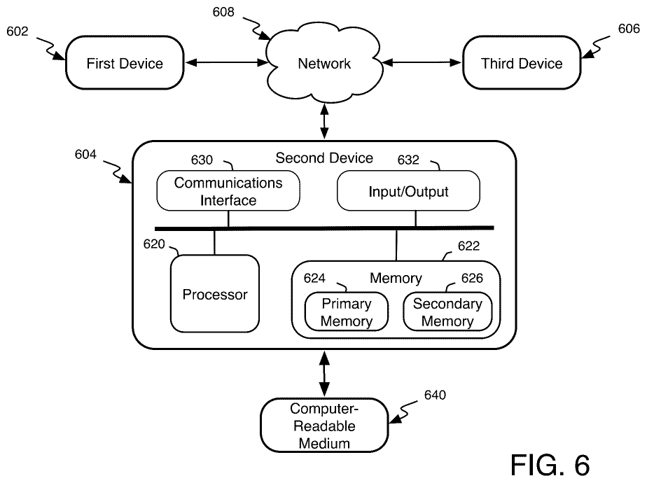

In one example embodiment, as shown in FIG. 6, a system embodiment may comprise a local network (e.g., device 604 and medium 640) and/or another type of network, such as a computing and/or communications network. For purposes of illustration, therefore, FIG. 6 shows an embodiment 600 of a system that may be employed to implement either type or both types of networks. Network 608 may comprise one or more network connections, links, processes, services, applications, and/or resources to facilitate and/or support communications, such as an exchange of communication signals, for example, between a computing device, such as 602, and another computing device, such as 606, which may, for example, comprise one or more client computing devices and/or one or more server computing device. By way of example, but not limitation, network 608 may comprise wireless and/or wired communication links, telephone and/or telecommunications systems, Wi-Fi networks, Wi-MAX networks, the Internet, a local area network (LAN), a wide area network (WAN), or any combinations thereof.

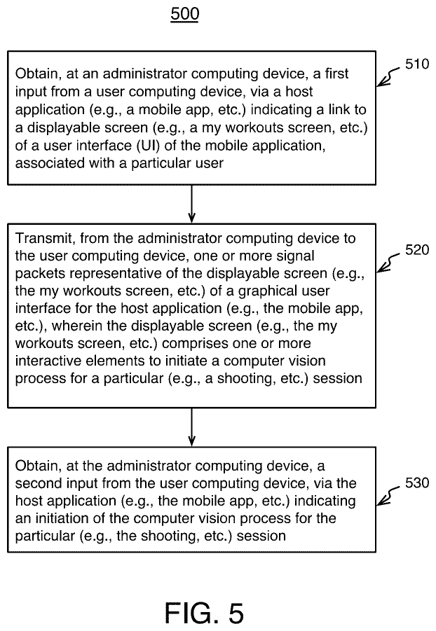

Example devices in FIG. 6 may comprise features, for example, of a client computing device and/or a server computing device, in an embodiment. It is further noted that the term computing device, in general, whether employed as a client and/or as a server, or otherwise, refers at least to a processor and a memory connected by a communication bus. Likewise, in the context of the present patent application at least, this is understood to refer to sufficient structure within the meaning of 35 USC § 112(f) so that it is specifically intended that 35 USC § 112(f) not be implicated by use of the term “computing device” and/or similar terms; however, if it is determined, for some reason not immediately apparent, that the foregoing understanding cannot stand and that 35 USC § 112(f), therefore, necessarily is implicated by the use of the term “computing device” and/or similar terms, then, it is intended, pursuant to that statutory section, that corresponding structure, material and/or acts for performing one or more functions be understood and be interpreted to be described at least in FIGS. 1-5 and in the text associated at least with the foregoing figure(s) of the present patent application.

Referring now to FIG. 6, in an embodiment, first and third devices 602 and 606 may be capable of rendering a graphical user interface (GUI) for a network device and/or a computing device, for example, so that a user-operator may engage in system use. Device 604 may potentially serve a similar function in this illustration. Likewise, in FIG. 6, computing device 602 (‘first device’ in figure) may interface with computing device 604 (‘second device’ in figure), which may, for example, also comprise features of a client computing device and/or a server computing device, in an embodiment. Processor (e.g., processing device) 620 and memory 622, which may comprise primary memory 624 and secondary memory 626, may communicate by way of a communication bus 615, for example. The term “computing device,” in the context of the present patent application, refers to a system and/or a device, such as a computing apparatus, that includes a capability to process (e.g., perform computations) and/or store digital content, such as electronic files, electronic documents, measurements, text, images, video, audio, sensor content, etc. in the form of signals and/or states. Thus, a computing device, in the context of the present patent application, may comprise hardware, software, firmware, or any combination thereof (other than software per se). Computing device 604, as depicted in FIG. 6, is merely one example, and claimed subject matter is not limited in scope to this particular example.

For one or more embodiments, a device, such as a computing device and/or networking device, may comprise, for example, any of a wide range of digital electronic devices, including, but not limited to, desktop and/or notebook computers, high-definition televisions, game consoles, satellite television receivers, cellular telephones, tablet devices, wearable devices, personal digital assistants, mobile audio and/or video playback and/or recording devices, Internet of Things (IOT) type devices, endpoint and/or sensor nodes, gateway devices, streaming devices, or any combination of the foregoing. Further, unless specifically stated otherwise, a process as described, such as with reference to flow diagrams and/or otherwise, may also be executed and/or affected, in whole or in part, by a computing device and/or a network device. A device, such as a computing device and/or network device, may vary in terms of capabilities and/or features. Claimed subject matter is intended to cover a wide range of potential variations. For example, a device may include a numeric keypad and/or other display of limited functionality, such as a monochrome liquid crystal display (LCD) for displaying text, for example. In contrast, however, as another example, a web-enabled device may include a physical and/or a virtual keyboard, mass storage, one or more accelerometers, one or more gyroscopes, global positioning system (GPS) and/or other location-identifying type capability, and/or a display with a higher degree of functionality, such as a touch-sensitive color 2D or 3D display, for example.

As suggested previously, communications between a computing device and/or a network device and a wireless network may be in accordance with known and/or to be developed network protocols including, for example, global system for mobile communications (GSM), enhanced data rate for GSM evolution (EDGE), 802.11b/g/n/h, etc., and/or worldwide interoperability for microwave access (WiMAX). A computing device and/or a networking device may also have a subscriber identity module (SIM) card, which, for example, may comprise a detachable or embedded smart card that is able to store subscription content of a user, and/or is also able to store a contact list. It is noted, however, that a SIM card may also be electronic, meaning that is may simply be stored in a particular location in memory of the computing and/or networking device. A user may own the computing device and/or network device or may otherwise be a user, such as a primary user, for example. A device may be assigned an address by a wireless network operator, a wired network operator, and/or an Internet Service Provider (ISP). For example, an address may comprise a domestic or international telephone number, an Internet Protocol (IP) address, and/or one or more other identifiers. In other embodiments, a computing and/or communications network may be embodied as a wired network, wireless network, or any combinations thereof.

A computing and/or network device may include and/or may execute a variety of now known and/or to be developed operating systems, derivatives and/or versions thereof, including computing device operating systems, such as Windows, macOS, iOS, Linux, and/or the like, and/or mobile device operating systems, such as iOS, Android, Windows Mobile, and/or the like. A computing device and/or network device may include and/or may execute a variety of possible applications, such as a client software application enabling communication with other devices. For example, one or more messages (e.g., content) may be communicated, such as via one or more protocols, now known and/or later to be developed, suitable for communication of email, short message service (SMS), and/or multimedia message service (MMS), including via a network, such as a social network, formed at least in part by a portion of a computing and/or communications network, including, but not limited to, Facebook, LinkedIn, Twitter, and/or Flickr, to provide only a few examples. A computing and/or network device may also include executable computer instructions to process and/or communicate digital content, such as, for example, textual content, digital multimedia content, sensor content, and/or the like. A computing and/or network device may also include executable computer instructions to perform a variety of possible tasks, such as browsing, searching, playing various forms of digital content, including locally stored and/or streamed video, and/or games such as, but not limited to, fantasy sports leagues. The foregoing is provided merely to illustrate that claimed subject matter is intended to include a wide range of possible features and/or capabilities.

In FIG. 6, computing device 602 may provide one or more sources of executable computer instructions in the form physical states and/or signals (e.g., stored in memory states), for example. Computing device 602 may communicate with computing device 604 by way of a network connection, such as via network 608, for example. As previously mentioned, a connection, while physical, may not necessarily be tangible. Although computing device 604 of FIG. 6 shows various tangible, physical components, claimed subject matter is not limited to a computing devices having only these tangible components as other implementations and/or embodiments may include alternative arrangements that may comprise additional tangible components or fewer tangible components, for example, that function differently while achieving similar results. Rather, examples are provided merely as illustrations. It is not intended that claimed subject matter be limited in scope to illustrative examples.

Memory 622 may comprise any non-transitory storage mechanism. Memory 622 may comprise, for example, primary memory 624 and secondary memory 626, additional memory circuits, mechanisms, or combinations thereof may be used. Memory 622 may comprise, for example, random access memory, read only memory, etc., such as in the form of one or more storage devices and/or systems, such as, for example, a disk drive including an optical disc drive, a tape drive, a solid-state memory drive, etc., just to name a few examples.

Memory 622 may be utilized to store a program of executable computer instructions. For example, processor 620 may fetch executable instructions from memory and proceed to execute the fetched instructions. Memory 622 may also comprise a memory controller for accessing device readable-medium 640 that may carry and/or make accessible digital content, which may include code, and/or instructions, for example, executable by processor 620 and/or some other device, such as a controller, as one example, capable of executing computer instructions, for example. Under direction of processor 620, a non-transitory memory, such as memory cells storing physical states (e.g., memory states), comprising, for example, a program of executable computer instructions, may be executed by processor 620 and able to generate signals to be communicated via a network, for example, as previously described. Generated signals may also be stored in memory, also previously suggested.

Memory 622 may store electronic files and/or electronic documents, such as relating to one or more users, and may also comprise a computer-readable medium that may carry and/or make accessible content, including code and/or instructions, for example, executable by processor 620 and/or some other device, such as a controller, as one example, capable of executing computer instructions, for example. As previously mentioned, the term electronic file and/or the term electronic document are used throughout this document to refer to a set of stored memory states and/or a set of physical signals associated in a manner so as to thereby form an electronic file and/or an electronic document. That is, it is not meant to implicitly reference a particular syntax, format and/or approach used, for example, with respect to a set of associated memory states and/or a set of associated physical signals. It is further noted an association of memory states, for example, may be in a logical sense and not necessarily in a tangible, physical sense. Thus, although signal and/or state components of an electronic file and/or electronic document, are to be associated logically, storage thereof, for example, may reside in one or more different places in a tangible, physical memory, in an embodiment.

Algorithmic descriptions and/or symbolic representations are examples of techniques used by those of ordinary skill in the signal processing and/or related arts to convey the substance of their work to others skilled in the art. An algorithm is, in the context of the present patent application, and generally, is considered to be a self-consistent sequence of operations and/or similar signal processing leading to a desired result. In the context of the present patent application, operations and/or processing involve physical manipulation of physical quantities. Typically, although not necessarily, such quantities may take the form of electrical and/or magnetic signals and/or states capable of being stored, transferred, combined, compared, processed and/or otherwise manipulated, for example, as electronic signals and/or states making up components of various forms of digital content, such as signal measurements, text, images, video, audio, etc.

It has proven convenient at times, principally for reasons of common usage, to refer to such physical signals and/or physical states as bits, values, elements, parameters, symbols, characters, terms, numbers, numerals, measurements, content and/or the like. It should be understood, however, that all of these and/or similar terms are to be associated with appropriate physical quantities and are merely convenient labels. Unless specifically stated otherwise, as apparent from the preceding discussion, it is appreciated that throughout this specification discussions utilizing terms such as “processing,” “computing,” “calculating,” “determining”, “establishing”, “obtaining”, “identifying”, “selecting”, “generating”, and/or the like may refer to actions and/or processes of a specific apparatus, such as a special purpose computer and/or a similar special purpose computing and/or network device. In the context of this specification, therefore, a special purpose computer and/or a similar special purpose computing and/or network device is capable of processing, manipulating and/or transforming signals and/or states, typically in the form of physical electronic and/or magnetic quantities, within memories, registers, and/or other storage devices, processing devices, and/or display devices of the special purpose computer and/or similar special purpose computing and/or network device. In the context of this particular patent application, as mentioned, the term “specific apparatus” therefore includes a general purpose computing and/or network device, such as a general purpose computer, once it is programmed to perform particular functions, such as pursuant to program software instructions.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and/or storage of charge or a release of stored charge. Likewise, in other memory devices, a change of state may comprise a physical change, such as a transformation in magnetic orientation. Likewise, a physical change may comprise a transformation in molecular structure, such as from crystalline form to amorphous form or vice-versa. In still other memory devices, a change in physical state may involve quantum mechanical phenomena, such as, superposition, entanglement, and/or the like, which may involve quantum bits (qubits), for example. The foregoing is not intended to be an exhaustive list of all examples in which a change in state from a binary one to a binary zero or vice-versa in a memory device may comprise a transformation, such as a physical, but non-transitory, transformation. Rather, the foregoing is intended as illustrative examples.

Referring again to FIG. 6, processor 620 may comprise one or more circuits, such as digital circuits, to perform at least a portion of a computing procedure and/or process. By way of example, but not limitation, processor 620 may comprise one or more processors, such as controllers, microprocessors, microcontrollers, application specific integrated circuits, digital signal processors (DSPs), graphics processing units (GPUs), neural network processing units (NPUs), programmable logic devices, field programmable gate arrays, the like, or any combination thereof. In various implementations and/or embodiments, processor 620 may perform signal processing, typically substantially in accordance with fetched executable computer instructions, such as to manipulate signals and/or states, to construct signals and/or states, etc., with signals and/or states generated in such a manner to be communicated and/or stored in memory, for example.

FIG. 6 also illustrates device 604 as including a component 632 operable with input/output devices, for example, so that signals and/or states may be appropriately communicated between devices, such as device 604 and an input device and/or device 604 and an output device. A user may make use of an input device, such as a computer mouse, stylus, track ball, keyboard, and/or any other similar device capable of receiving user actions and/or motions as input signals. Likewise, for a device having speech to text capability, a user may speak to a device to generate input signals. A user may make use of an output device, such as a display, a printer, etc., and/or any other device capable of providing signals and/or generating stimuli for a user, such as visual stimuli, audio stimuli and/or other similar stimuli.

According to an embodiment, a neural network may comprise a graph comprising nodes to model neurons in a brain. In this context, a “neural network” as referred to herein means an architecture of a processing device defined and/or represented by a graph including operators (represented by nodes in the graph) to model neurons that process input signals to generate output signals, and tensors (represented by edges in the graph) connecting the operators to represent input and/or output signal paths between and/or among operators (represented by nodes the graph). In particular implementations, a neural network may comprise a biological neural network, made up of real biological neurons, or an artificial neural network, made up of artificial neurons, for solving artificial intelligence (AI) problems, for example. In an implementation, such an artificial neural network may be implemented by one or more computing devices such as computing devices including a central processing unit (CPU), graphics processing unit (GPU), digital signal processing (DSP) unit and/or neural processing unit (NPU), just to provide a few examples. In a particular implementation, neural network weights and/or numerical coefficients associated with edges to represent input and/or output paths may reflect gains to be applied and/or whether an associated connection between connected nodes is to be excitatory (e.g., weight with a positive value) or inhibitory connections (e.g., weight with negative value). In an example implementation, a neuron may apply a neural network weight to input signals, and sum weighted input signals to generate a linear combination.

According to an embodiment, edges in a neural network connecting nodes may model synapses capable of transmitting signals (e.g., represented by real number values) between neurons. Responsive to receipt of such a signal, a node/neural may perform some computation to generate an output signal (e.g., to be provided to another node in the neural network connected by an edge). Such an output signal may be based, at least in part, on one or more weights and/or numerical coefficients associated with the node and/or edges providing the output signal. For example, such a weight may increase or decrease a strength of an output signal. In a particular implementation, such weights and/or numerical coefficients may be adjusted and/or updated as a machine learning process progresses. In an implementation, transmission of an output signal from a node in a neural network may be inhibited if a strength of the output signal does not exceed a threshold value.

FIG. 7 is a schematic diagram of a neural network 700 formed in “layers” in which an initial layer is formed by nodes 702 and a final layer is formed by nodes 706. Neural network (NN) 700 may include an intermediate layer formed by nodes 704. Edges shown between nodes 702 and 704 illustrate signal flow from an initial layer to an intermediate layer. Likewise, edges shown between nodes 704 and 706 illustrate signal flow from an intermediate layer to a final layer. While neural network 700 shows a single intermediate layer formed by nodes 704, it should be understood that other implementations of a neural network may include multiple intermediate layers formed between an initial layer and a final layer.

According to an embodiment, a node 702, 704 and/or 706 may process input signals (e.g., received on one or more incoming edges) to provide output signals (e.g., on one or more outgoing edges) according to operators defined at associated nodes. At a node of neural network 700, a set of one or more operations associated with the node may map one or more input signals to one or more output signals. In a particular implementation, such a set of one or more operations may be defined based, at least in part, on a weight associated with the node. One class of operators defined at nodes of neural network 700 may comprise matrix multiplication-like operators such as, for example, convolutions, depth-wise convolutions, matrix multiplies, fully connected, batched matrix multiply and so forth, which may be further defined by associated weights. Another class of operators defined at nodes of neural network 700 may comprise “activation functions” constructed as unary element-wise operators that introduce a non-linearity. Examples of such activation functions may include, for example, logistic (e.g., sigmoid and/or soft step), hyperbolic tangent, rectified linear unit, Gaussian error linear unit, Softplus, exponential linear unit, scaled exponential linear unit, leaky rectified linear unit, parametric rectified linear unit, sigmoid linear unit, Swish, Mish, Gaussian and/or growing cosine unit functions. Such activation functions may or may not be defined by trainable weights. A parametric rectified linear unit function, in particular, may be defined by a trainable alpha value. Additional element-wise operators defined at nodes of neural network 700 may include multiply, add, subtract, negate, select and so forth. Nodes in neural network 700 may also represent layout-like operators, such as Transpose, Reshape, Slice, Concat, Tile, just to provide a few examples.

Additionally, an “input value” as referred to herein means a value provided as an input parameter and/or signal to one or more operators defined and/or represented by a node in a neural network. Likewise, an “output value” as referred to herein means an output value provided by one or more operators defined and/or represented by a node of a neural network. In a particular implementation, an output value may be computed and/or generated according to one or more operators based on and/or responsive to one or more input values received at a node. In a particular implementation, an input value and/or output value may be structured, dimensioned and/or formatted as “tensors”. Thus, in this context, an “input tensor” as referred to herein means an expression of one or more input values according to a particular structure, dimension and/or format. Likewise in this context, an “output tensor” as referred to herein means an expression of one or more output values according to a particular structure, dimension and/or format.

According to an embodiment, neural network 700 may be characterized as having a particular structure or topology based on, for example, a number of layers, number of nodes in each layers, operators implemented at each node, quantization of weights and quantization of input/output tensors. Neural network 700 may be further characterized by weights to be assigned to nodes to affect operators at respective nodes. During execution, neural network 700 may be characterized as having a particular state or “intermediate state” determined based on values/signals computed by nodes (e.g., as tensor values to be provided to nodes in a subsequent layer of nodes and/or an output tensor).

In particular implementations, neural networks may enable improved results in a wide range of tasks, including image recognition, speech recognition, just to provide a couple of example applications. To enable performing such tasks, features of a neural network (e.g., nodes, edges, weights, layers of nodes and edges) may be structured and/or configured to form “filters” that may have a measurable/numerical state such as a value of an output signal. Such a filter may comprise nodes and/or edges arranged in “paths” and are to be responsive to sensor observations provided as input signals. In an implementation, a state and/or output signal of such a filter may indicate and/or infer detection of a presence or absence of a feature in an input signal.

In particular implementations, intelligent computing devices to perform functions supported by neural networks may comprise a wide variety of stationary and/or mobile devices, such as, for example, automobile sensors, biochip transponders, heart monitoring implants, Internet of things (IoT) devices, kitchen appliances, locks or like fastening devices, solar panel arrays, home gateways, smart gauges, robots, financial trading platforms, smart telephones, cellular telephones, security cameras, wearable devices, thermostats, Global Positioning System (GPS) transceivers, personal digital assistants (PDAs), virtual assistants, laptop computers, personal entertainment systems, tablet personal computers (PCs), PCs, personal audio or video devices, personal navigation devices, just to provide a few examples.

According to an embodiment, a neural network may be structured in layers such that a node in a particular neural network layer may receive output signals from one or more nodes in an upstream layer in the neural network, and provide an output signal to one or more nodes in a downstream layer in the neural network. One specific class of layered neural networks may comprise a convolutional neural network (CNN) or space invariant artificial neural networks (SIANN) that enable deep learning. Such CNNs and/or SIANNs may be based, at least in part, on a shared-weight architecture of a convolution kernels that shift over input features and provide translation equivariant responses. Such CNNs and/or SIANNs may be applied to image and/or video recognition, recommender systems, image classification, image segmentation, medical image analysis, natural language processing (e.g., medical records processing), brain-computer interfaces, financial time series, just to provide a few examples.

Another class of layered neural network may comprise a recursive neural network (RNN) that is a class of neural networks in which connections between nodes form a directed cyclic graph along a temporal sequence. Such a temporal sequence may enable modeling of temporal dynamic behavior. In an implementation, an RNN may employ an internal state (e.g., memory) to process variable length sequences of inputs. This may be applied, for example, to tasks such as unsegmented, connected handwriting recognition or speech recognition, just to provide a few examples. In particular implementations, an RNN may emulate temporal behavior using finite impulse response (FIR) or infinite impulse response (IIR) structures. An RNN may include additional structures to control stored states of such FIR and IIR structures to be aged. Structures to control such stored states may include a network or graph that incorporates time delays and/or has feedback loops, such as in long short-term memory networks (LSTMs) and gated recurrent units.

According to an embodiment, output signals of one or more neural networks (e.g., taken individually or in combination) may at least in part, define a “predictor” to generate prediction values associated with some observable and/or measurable phenomenon and/or state. In an implementation, a neural network may be “trained” to provide a predictor that is capable of generating such prediction values based on input values (e.g., measurements and/or observations) optimized according to a loss function. For example, a training process may employ backpropagation techniques. “Backpropagation,” as referred to herein, is to mean a process of fitting parameters of a trained inference model such a model comprising one or more neural networks. In fitting parameters of a neural network, for example, backpropagation is to compute a gradient of a loss function with respect to the weights of the neural network. Based on such a computed gradient of a loss function, weights may be updated so as to minimize and/or reduce such a loss function. In one particular implementation, a gradient descent of a loss function, or variants such as stochastic gradient descent of a loss function, may be used. In training parameters of a neural network, backpropagation may comprise computing a gradient of a loss function with respect to individual weights by the chain rule, computing a gradient one layer at a time, iterating backward from the last layer to avoid redundant calculations of intermediate terms in the chain rule, for example. It should be understood, however, that this is merely an example of how a process of backpropagation may be applied, and claimed subject matter is not limited in this respect. In particular implementations, backpropagation may be used to iteratively update neural network weights to be associated with nodes and/or edges of a neural network based, at least in part on “training sets.” Such training sets may include training measurements and/or observations to be supplied as input values that are paired with “ground truth” observations. Based on a comparison of such ground truth observations and associated prediction values generated based on such input values in a training process, weights may be updated according to a loss function using backpropagation.

FIG. 8 is a flow diagram of an aspect of a training operation employing backpropagation to train parameters for a feedforward neural network, according to an embodiment. It should be understood, however, that this is merely an example of a type of neural network that may be trained using backpropagation, and that similar backpropagation techniques may be applied to train parameters of other types of neural networks without deviating from claimed subject matter. Training sets may be provided to such a training operation as pairs of vectors (x,y) where x is an input vector and y is a corresponding ground truth label. Input vector x may be provided as an input tensor to a first hidden layer 804 to produce an output vector h⁽¹⁾, which is provided as an input to a second hidden layer 806 to provide an output vector h⁽²⁾. An inference and/or prediction ŷ may be computed based, at least in part, on the output vector h⁽²⁾. A loss function C may be computed at 802 based, at least in part, on inference and/or prediction ŷ and ground truth label y.

In the particular embodiment of FIG. 8, inference and/or prediction y, and output vectors h⁽¹⁾ and h⁽²⁾ may be modelled as follows:

$${h}^{( 1 )} = {g}^{( 1 )} ( {W}^{( 1 ) T} x + {b}^{( 1 )} )$$

$${h}^{( 2 )} = {g}^{( 2 )} ( {W}^{( 2 ) T} {h}^{( 1 )} + {b}^{( 2 )} )$$

$$\hat{y} ( x ) = {W}^{( 3 ) T} {h}^{( 2 )} + {b}^{( 3 )} ,$$

where:
g^{(t)} is an activation function applied at nodes in hidden layer i;
w⁽ⁱ⁾ is a matrix of weights such that weight

$${W}_{jk}^{( i )}$$

is to be applied at an edge going from node j in layer i−1 to node k in hidden layer i; and
b⁽ⁱ⁾ is a bias matrix applied at hidden layer i.

In a particular implementation in which a feedforward neural network includes three or more hidden layers, computation of ŷ(x) may be generalized as follows:

$$\hat{y} ( x ) = {W}^{( N ) T} {h}^{( N - 1 )} + {b}^{( N )} .$$

Loss function C (y,ŷ) may be computed according to any one of several formulations of a loss function as described above. In a particular implementation, C(y,ŷ) may be differentiable such that

$$\frac{\partial C}{\partial {W}_{jk}^{( i )}}$$

may be determined using the chain rule and may be computed for any weight

$${W}_{jk}^{( i )} .$$

According to an embodiment, values for W⁽ⁱ⁾ may be determined iteratively for training sets (x,y) using a gradient descent technique.

In the preceding description, various aspects of claimed subject matter have been described. For purposes of explanation, specifics, such as amounts, systems and/or configurations, as examples, were set forth. In other instances, well-known features were omitted and/or simplified so as not to obscure claimed subject matter. While certain features have been illustrated and/or described herein, many modifications, substitutions, changes and/or equivalents will now occur to those skilled in the art. It is, therefore, to be understood that the appended claims are intended to cover all modifications and/or changes as fall within claimed subject matter.

What is claimed is:

1. A method, comprising:
obtaining, at an administrator computing device, a first input from a user computing device, via a host application indicating a selection of a link to a displayable screen associated with a particular user;
transmitting, from the administrator computing device to the user computing device, one or more signal packets representative of the displayable screen of a graphical user interface for the host application, wherein the displayable screen comprises one or more interactive elements to initiate a computer vision process for a particular session; and
obtaining, at the administrator computing device, a second input from the user computing device, via the host application indicating an initiation of the computer vision process for the particular session.

2. The method of claim 1, further comprising obtaining, at the administrator computing device, a third input from the user computing device specifying a period of time for the vision shooting session.

3. The method of claim 2, further comprising transmitting, from the administrator computing device to the user computing device, one or more signal packets representative of one or more screens pertaining to a calibration operation for the vision shooting session.

4. The method of claim 3, wherein the calibration operation comprises:
obtaining video and/or image content of a portion of a basketball court via a camera of the user computing device;
displaying a calibration screen depicting the portion of the basketball court via the graphical user interface of the mobile app, wherein the calibration screen comprises a representation of a 3-point line and a representation of a basketball hoop;
calculating measurements with respect to the 3-point line and the basketball hoop; and
determining whether the 3-point line and the basketball hoop have been accurately detected via the video and/or image content of the portion of the basketball court.

5. The method of claim 4, further comprising causing to be displayed on the user computing device an indication of a successful calibration operation.

6. The method of claim 5, further comprising obtaining from the user computing device a further input indicating selection of a “start recording” button displayed to the particular user via the graphical user interface.

7. The method of claim 6, further comprising displaying indications of shots made and/or shots missed via the graphical user interface in real-time or near real-time.

8. The method of claim 7, further comprising obtaining from the user computing device a further input indicating selection of a “stop recording” button displayed to the particular user via the graphical user interface.

9. The method of claim 8, further comprising displaying a vision shooting session results screen to the particular user via the graphical user interface, wherein the vision shooting session results screen includes interactive elements for options to keep session data and/or to delete session data.

10. The method of claim 9, wherein the my workouts screen comprises an interactive shot chart depicting multiple selectable shooting zones and/or further depicting shooting statistics pertaining to the multiple selectable shootings zones for one or more stored vision shooting sessions.

11. The method of claim 10, wherein the vision shooting session results screen includes an interactive element for an option to alert the mobile app of inaccurate statistics related to the vision shooting session.

12. The method of claim 11, wherein the particular session screen comprises an interactive element for an option to view a screen depicting stored vision shooting sessions.

13. A method, comprising:
obtaining, at an administrator computing device, a first input from a user computing device, via a mobile app, indicating a selection of a link to a home screen pertaining to a particular group;
transmitting, from the administrator computing device to the user computing device, one or more signal packets representative of the group home screen of a graphical user interface for the mobile app, wherein the group home screen comprises one or more interactive elements to initiate a vision team shooting session; and
obtaining, at the administrator computing device, a second input from the user computing device, via the mobile app, indicating an initiation of the vision team shooting session.

14. The method of claim 13, wherein the group home screen comprises an interactive shot chart depicting multiple selectable shooting zones and/or further depicting shooting statistics pertaining to the multiple selectable shootings zones for one or more stored vision team shooting sessions.

15. The method of claim 14, wherein the group home screen comprises an interactive element for an option to view a screen depicting vision shooting sessions for individual members of a group.

16. The method of claim 15, wherein the screen depicting vision shooting sessions for individual members of the group includes one or more leaderboards for one or more statistical categories pertaining to the vision shooting sessions for the individual members of the group.

17. The method of claim 16, wherein the screen depicting vision shooting sessions for individual members of the group depicts interactive elements to select a particular vision shooting session for a particular member of the group.

18. The method of claim 17, wherein the screen depicting vision shooting sessions for individual members of the group includes one or more interactive elements for options to view results for one or more vision shooting sessions for the a specified member of the group.

19. The method of claim 18, wherein the group home screen comprises interactive elements for options to perform Ballogy Skills Assessments, to view stored vision team shooting sessions, and/or to view one or more workouts.

20. The method of claim 19, wherein the group home screen comprises interactive elements for options to create a new workout and/or assign an existing workout.

21. An apparatus, comprising: at least one processor of an administrator computing device to:
obtain a first input from a user computing device, via a mobile app executed at the user computing device, indicating a selection of a link to a my workouts screen associated with a particular user;
transmit, from the administrator computing device to the user computing device, one or more signal packets representative of the my workouts screen of a graphical user interface for the mobile app, wherein the my workouts screen comprises one or more interactive elements to initiate a vision shooting session; and
obtain, at the administrator computing device, a second input from the user computing device, via the mobile app, indicating an initiation of a vision shooting session.

22. The apparatus of claim 21, wherein the at least one processor is further to obtain, at the administrator computing device, a third input from the user computing device specifying a period of time for the vision shooting session.

23. The apparatus of claim 22, wherein the at least one processor is further to transmit, from the administrator computing device to the user computing device, one or more signal packets representative of one or more screens pertaining to a calibration operation for the vision shooting session.

24. The apparatus of claim 23, wherein the calibration operation comprises:
obtaining video and/or image content of a portion of a basketball court via a camera of the user computing device;
displaying a calibration screen depicting the portion of the basketball court via the graphical user interface of the mobile app, wherein the calibration screen comprises a representation of a 3-point line and a representation of a basketball hoop;
calculating measurements with respect to the 3-point line and the basketball hoop; and
determining whether the 3-point line and the basketball hoop have been accurately detected via the video and/or image content of the portion of the basketball court.

25. The apparatus of claim 24, further comprising causing to be displayed on the user computing device an indication of a successful calibration operation.

26. The apparatus of claim 25, wherein the at least one processor is further to obtain from the user computing device a further input indicating selection of a “start recording” button displayed to the particular user via the graphical user interface.

27. The apparatus of claim 26, wherein the user computing device is to display indications of shots made and/or shots missed via the graphical user interface in real-time or near real-time.

28. The apparatus of claim 27, wherein the at least one processor is further to obtain from the user computing device a further input indicating selection of a “stop recording” button displayed to the particular user via the graphical user interface.

29. The apparatus of claim 28, wherein the at least one processor is further to display a vision shooting session results screen to the particular user via the graphical user interface, wherein the vision shooting session results screen includes interactive elements for options to keep session data and/or to delete session data.

30. The apparatus of claim 29, wherein the my workouts screen comprises an interactive shot chart depicting multiple selectable shooting zones and/or further depicting shooting statistics pertaining to the multiple selectable shootings zones for one or more stored vision shooting sessions.

31. The apparatus of claim 30, wherein the vision shooting session results screen includes an interactive element for an option to alert the mobile app of inaccurate statistics related to the vision shooting session.

32. The apparatus of claim 31, wherein the my workouts screen comprises an interactive element for an option to view a screen depicting stored vision shooting sessions.

33. An apparatus, comprising: at least one processor of an administrator computing device to:
obtain, at an administrator computing device, a first input from a user computing device, via a mobile app, indicating a selection of a link to a home screen pertaining to a particular group;
transmit, from the administrator computing device to the user computing device, one or more signal packets representative of the group home screen of a graphical user interface for the mobile app, wherein the group home screen comprises one or more interactive elements to initiate a vision team shooting session; and
obtain, at the administrator computing device, a second input from the user computing device, via the mobile app, indicating an initiation of the vision team shooting session.

34. The apparatus of claim 33, wherein the group home screen comprises an interactive shot chart depicting multiple selectable shooting zones and/or further depicting shooting statistics pertaining to the multiple selectable shootings zones for one or more stored vision team shooting sessions.

35. The apparatus of claim 34, wherein the group home screen comprises an interactive element for an option to view a screen depicting vision shooting sessions for individual members of a group.

36. The apparatus of claim 35, wherein the screen depicting vision shooting sessions for individual members of the group includes one or more leaderboards for one or more statistical categories pertaining to the vision shooting sessions for the individual members of the group.

37. The apparatus of claim 36, wherein the screen depicting vision shooting sessions for individual members of the group depicts interactive elements to select a particular vision shooting session for a particular member of the group.

38. The apparatus of claim 37, wherein the screen depicting vision shooting sessions for individual members of the group includes one or more interactive elements for options to view results for one or more vision shooting sessions for the a specified member of the group.

39. The apparatus of claim 38, wherein the group home screen comprises interactive elements for options to perform skills assessments, to view stored vision team shooting sessions, and/or to view one or more workouts.

40. The apparatus of claim 39, wherein the group home screen comprises interactive elements for options to create a new workout and/or assign an existing workout.

41. An article, comprising: a non-transitory computer-readable storage medium having stored thereon instructions executable by at least one processor of an administrator computing device to:
obtain a first input from a user computing device, via a mobile app executed at the user computing device, indicating a selection of a link to a my workouts screen associated with a particular user;
transmit, from the administrator computing device to the user computing device, one or more signal packets representative of the my workouts screen of a graphical user interface for the mobile app, wherein the my workouts screen comprises one or more interactive elements to initiate a vision shooting session; and
obtain, at the administrator computing device, a second input from the user computing device, via the mobile app, indicating an initiation of a vision shooting session.

42. The article of claim 41, wherein the at least one processor is further to obtain, at the administrator computing device, a third input from the user computing device specifying a period of time for the vision shooting session.

43. The article of any of claim 42, wherein the at least one processor is further to transmit, from the administrator computing device to the user computing device, one or more signal packets representative of one or more screens pertaining to a calibration operation for the vision shooting session.

44. The article of claim 43, wherein the calibration operation comprises:
obtaining video and/or image content of a portion of a basketball court via a camera of the user computing device;
displaying a calibration screen depicting the portion of the basketball court via the graphical user interface of the mobile app, wherein the calibration screen comprises a representation of a 3-point line and a representation of a basketball hoop;
calculating measurements with respect to the 3-point line and the basketball hoop; and
determining whether the 3-point line and the basketball hoop have been accurately detected via the video and/or image content of the portion of the basketball court.

45. The article of claim 44, wherein the at least one processor is further to cause to be displayed on the user computing device an indication of a successful calibration operation.

46. The article of claim 45, wherein the at least one processor is further to obtain from the user computing device a further input indicating selection of a “start recording” button displayed to the particular user via the graphical user interface.

47. The article of claim 46, wherein the user computing device is to display indications of shots made and/or shots missed via the graphical user interface in real-time or near real-time.

48. The article of claim 47, wherein the at least one processor is further to obtain from the user computing device a further input indicating selection of a “stop recording” button displayed to the particular user via the graphical user interface.

49. The article of claim 48, wherein the at least one processor is further to display a vision shooting session results screen to the particular user via the graphical user interface, wherein the vision shooting session results screen includes interactive elements for options to keep session data and/or to delete session data.

50. The article of claim 49, wherein the my workouts screen comprises an interactive shot chart depicting multiple selectable shooting zones and/or further depicting shooting statistics pertaining to the multiple selectable shootings zones for one or more stored vision shooting sessions.

51. The article of claim 50, wherein the vision shooting session results screen includes an interactive element for an option to alert the mobile app of inaccurate statistics related to the vision shooting session.

52. The article of claim 51, wherein the my workouts screen comprises an interactive element for an option to view a screen depicting stored vision shooting sessions.

53. An apparatus, comprising: at least one processor of an administrator computing device to:
obtain, at an administrator computing device, a first input from a user computing device, via a mobile app, indicating a selection of a link to a home screen pertaining to a particular group;
transmit, from the administrator computing device to the user computing device, one or more signal packets representative of the group home screen of a graphical user interface for the mobile app, wherein the group home screen comprises one or more interactive elements to initiate a vision team shooting session; and
obtain, at the administrator computing device, a second input from the user computing device, via the mobile app, indicating an initiation of the vision team shooting session.

54. The article of claim 53, wherein the group home screen comprises an interactive shot chart depicting multiple selectable shooting zones and/or further depicting shooting statistics pertaining to the multiple selectable shootings zones for one or more stored vision team shooting sessions.

55. The article of claim 54, wherein the group home screen comprises an interactive element for an option to view a screen depicting vision shooting sessions for individual members of a group.

56. The article of claim 55, wherein the screen depicting vision shooting sessions for individual members of the group includes one or more leaderboards for one or more statistical categories pertaining to the vision shooting sessions for the individual members of the group.

57. The article of claim 56, wherein the screen depicting vision shooting sessions for individual members of the group depicts interactive elements to select a particular vision shooting session for a particular member of the group.

58. The article of claim 57, wherein the screen depicting vision shooting sessions for individual members of the group includes one or more interactive elements for options to view results for one or more vision shooting sessions for the a specified member of the group.

59. The article of claim 58, wherein the group home screen comprises interactive elements for options to perform Ballogy Skills Assessments, to view stored vision team shooting sessions, and/or to view one or more workouts.

60. The article of claim 59, wherein the group home screen comprises interactive elements for options to create a new workout and/or assign an existing workout.
